(19) United States
(12) Patent Application Publication
(10) Pub. No.: US 2025/0339462 A1
(43) Pub. Date: Nov. 6, 2025

(54) CANCER TREATMENT USING ULTRA-HIGH CONCENTRATION GASEOUS NITRIC OXIDE AND A CHECKPOINT INHIBITOR

(71) Applicant: Beyond Air, Inc., Garden City, NY (US)

(72) Inventors: Hila CONFINO, Be'er Ya'akov (IL); Matan GOLDSHTEIN, Yavne (IL); Amir AVNIEL, Har Adar (IL); Steven A. LISI, Rockville Centre, NY (US)

(21) Appl. No.: 18/987,013

(22) Filed: Dec. 19, 2024

Related U.S. Application Data
(63) Continuation of application No. PCT/US2023/026995, filed on Jul. 6, 2023.
(60) Provisional application No. 63/358,540, filed on Jul. 6, 2022.
(60) Provisional application No. 63/358,542, filed on Jul. 6, 2022.
(60) Provisional application No. 63/358,547, filed on Jul. 6, 2022.
(60) Provisional application No. 63/439,435, filed on Jan. 17, 2023.
(60) Provisional application No. 63/451,783, filed on Mar. 13, 2023.
(60) Provisional application No. 63/460,187, filed on Apr. 18, 2023.

(51) Int. Cl. A61K 33/00; A61K 9/00; A61K 31/7125; A61K 39/00; A61K 39/395; A61K 47/26; A61P 35/00

(52) U.S. Cl. CPC A61K 33/00; A61K 9/0019; A61K 31/7125; A61K 39/39558; A61K 47/26; A61P 35/00; A61K 2039/505

(57) ABSTRACT

Cancer treatment using ultra-high concentration gaseous nitric oxide (UNO) and a checkpoint inhibitor and, optionally, an immune adjuvant is provided. Additionally, UNO as a sensitizing treatment to checkpoint inhibitors is provided. Accordingly, there is provided a method of treating cancer in a subject in need thereof, the method comprising administering to the subject a therapeutically effective amount of UNO and a checkpoint inhibitor and, optionally, an immune adjuvant.

### RELATED APPLICATIONS

This application is a continuation of International Application No. PCT/US2023/026995, which designated the United States and was filed on Jul. 6, 2023 and published in English, which claims the benefit of U.S. Provisional Application No. 63/358,540 filed Jul. 6, 2022, U.S. Provisional Application No. 63/358,542 filed Jul. 6, 2022, U.S. Provisional Application No. 63/358,547 filed Jul. 6, 2022, U.S. Provisional Application No. 63/439,435 filed Jan. 17, 2023, U.S. Provisional Application No. 63/451,783 filed Mar. 13, 2023, and U.S. Provisional Application No. 63/460,187 filed Apr. 18, 2023. The entire contents of the above-referenced applications are incorporated by reference herein.

### FIELD OF THE INVENTION

The present invention, in some embodiments thereof, relates to cancer treatment using ultra-high concentration gaseous nitric oxide and a checkpoint inhibitor.

### BACKGROUND OF THE INVENTION

Cancer immunotherapy, including e.g., cell-based therapy, antibody therapy, cytokine or adjuvant therapy and vaccines, has emerged in the last couple of years as a promising strategy for treating various types of cancer. Thus, for example, modulation of the existing patient immune system through checkpoint inhibitors such as anti-CTLA-4, anti-PD-1 and anti-PD-L1 antibodies has led to durable remissions across a wide variety of different tumor types (e.g., Kamir J. et al. Nat Rev Cancer; volume 21 346-359).

To date the ability to successfully treat cancer using immune checkpoint antibodies has been very limited. The art has indicated numerous reasons and overall, the physicians' use of this tool is quite limited (De Miguel and Calvo Cancer Cell (2020) Sep. 14; 38(3):326-333).

In parallel, tumor ablation is a minimally invasive technique used in the treatment of solid tumors. There are several ablation methods, such as radiofrequency, microwave ablation, high intensity focused ultrasound ablation, laser ablation and cryoablation (Knavel, E. M and Brace, C. L. Tech Vase Interv Radiol 16(4), 192-200, 2013). Image-guided tumor ablation for early-stage hepatocellular carcinoma (HCC) is an accepted non-surgical treatment that provides local tumor control and favorable survival benefit (Kang, T. W and Rhim, H. Liver Cancer 4(3), 176-187, 2015). Local and in situ tumor ablation methods were shown to enhance anti-tumor immune responses resulting in the destruction of residual malignant cells in primary tumors and distant metastases [Keisari, Y. et al. Cancer Immunol. Immunother. 63, 1-9 (2014); Confino et al. Cancer Immunol Immunother 64(2) 191-199, 2015]. Notably, in contrast to surgical resection, using ablation, even with the bulk of the tumor destroyed, antigenic remnants persist in the tumor site/body. This aspect of ablation is responsible for its ability to trigger a systemic anti-tumor immune response.

Nitric oxide (NO) is a short-lived, endogenously produced gas that acts as a signaling molecule in the body (Thomas, D. D. Redox Biol 5, 225-233, 2015). Increasing evidence highlights its wide spectrum of action in different pathologic conditions, including cancer (Huerta, S. Futur. Sci. OA 1, FS044, 2015) and involvement in immune cell signaling against pathogens (Schairer et al. Virulence 3, 271-279, 2012).

Preclinical studies testing the effect of exogenously administered nitric oxide (NO) demonstrated its anti-cancer properties and suggested that NO may serve as a potent tumoricidal ablation agent. While NO at low doses may possess pro-oncogenic properties; at high doses, NO may have a role in cancer therapy either as a single agent or in combination with other antineoplastic compounds (e.g., Huerta S. Future Sci OA. 2015 Aug. 1; 1(1):FSO44; Vannini F. et al. Redox Biol. 2015 December; 6:334-343; Seabra A B et al. Eur J Pharmacol. 2018 May 5; 826:158-168; Alimoradi H. et al. Pharm Nanotechnol. 2019; 7(4):279-303; and Ning S. et al. Biochem Biophys Res Commun. 2014 May 9; 447(3):537-42). More specifically, high doses of NO were shown to promote oxidative/nitrosative stress and DNA damage. The generation of reactive nitric oxide species, including peroxynitrite can oxidize the DNA and induce single strand breaks. In addition, NO can induce cell death via both i) necrosis, and ii) apoptosis (Seabra A B and Durán N. Eur J Pharmacol. 2018 May 5; 826:158-168; Vannini F. et al. Redox Biol. 2015 December; 6:334-343).

Use of gaseous NO (gNO) in cancer treatment has been previously described in WO2021/105901; WO2021/105900; and WO2022/043931.

Additional background art includes:
Aizhang Xu et al. Cellular & Molecular Immunology (2019) 16:820-832;
Shu Wanga, et al. PNAS (2016), Oct. 31, 2016;
Stefania Cuzzubbo, et al. Front. Immunol. (2021) 17 Feb.

### SUMMARY OF THE INVENTION

According to an aspect of some embodiments of the present invention there is provided a method of treating cancer in a subject in need thereof, the method comprising administering to the subject a therapeutically effective amount of ultra-high concentration gaseous nitric oxide (UNO), e.g., between about 10,000 and 1,000,000 ppm, and a checkpoint inhibitor, thereby treating the cancer in the subject. According to an aspect of some embodiments of the present invention, UNO is used as a sensitizing treatment to the checkpoint inhibitor, wherein the UNO is used to upregulate the expression of a target immune checkpoint protein before the administration of the checkpoint inhibitor.

According to an aspect of some embodiments of the present invention, the method further comprises administering an immune adjuvant.

According to an aspect of some embodiments of the present invention, there is provided a combination of UNO and a checkpoint inhibitor, for use in treating cancer in a subject in need thereof.

According to an aspect of some embodiments of the present invention, the combination further comprises an immune adjuvant.

According to an aspect of some embodiments of the present invention, the at least one checkpoint inhibitor is selected from the group consisting of an anti-PD-1 antibody, an anti-PD-L1 antibody, an anti-CTLA-4 antibody, and an anti-LAG-3 antibody.

According to an aspect of some embodiments of the present invention, the anti-PD-1 antibody is one of pembrolizumab, nivolumab, cemiplimab, spartalizumab, sintilimab, tislelizumab, toripalimab, dostarlimab, JTX-4014, INCMGA00012, AMP-224, and AMP-514.

According to an aspect of some embodiments of the present invention, the anti-PD-L1 antibody is one of atezolizumab, durvalumab, avelumab, KN035, CK-301, AUNP12, CA-170 and BMS-986189.

According to an aspect of some embodiments of the present invention, the anti-CTLA-4 antibody is one of ipilimumab and tremelimumab.

According to an aspect of some embodiments of the present invention, the anti-LAG-3 antibody is relatlimab.

According to some embodiments of the invention, the UNO is administered locally.

According to some embodiments of the invention, local administration comprises intra-tumoral administration.

According to some embodiments of the invention, the UNO is administered at a dose of from about 10,000 ppm to about 1,000,000 ppm for a time period of from about 1 second to about 60 minutes at a volumetric flow of from about 0.00001 LPM to about 1 LPM.

According to some embodiments of the invention, the UNO is administered at a dose of from about 20,000 ppm to about 200,000 ppm, or from about 20,000 ppm to about 100,000 ppm.

According to some embodiments of the invention, the UNO is administered for a time period that ranges from about 1 second to about 10 minutes.

According to some embodiments of the invention, the UNO is administered at a volumetric flow of from about 0.001 LPM to about 0.5 LPM.

According to some embodiments of the invention, the checkpoint inhibitor is administered prior to the UNO.

According to some embodiments of the invention, the UNO is administered prior to the checkpoint inhibitor.

According to some embodiments of the invention, following administration of an effective amount of the UNO, the cells of the cancer express the immune checkpoint protein or a binding pair thereof.

According to some embodiments of the invention, the checkpoint inhibitor is administered every 2-7 days.

According to some embodiments of the invention, the checkpoint inhibitor is administered at least twice.

According to some embodiments of the invention, the cancer is refractory to treatment with the checkpoint inhibitor.

According to some embodiments of the invention, the immune checkpoint protein is selected from the group consisting of PD-1, PD-L1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to some embodiments of the invention, the immune checkpoint protein is selected from the group consisting of PD-1, PD-L1, CTLA-4, and LAG-3.

According to some embodiments of the invention, the checkpoint inhibitor is selected from the group consisting of an anti-PD-1 antibody, an anti-PD-L1 antibody, an anti-CTLA-4 antibody, and an anti-LAG-3 antibody.

According to some embodiments of the invention, the immune adjuvant is selected from the group consisting of mineral salt, aluminum salt, an organic adjuvant, emulsion, microparticle, liposome, saponin, cytokine, microbial component and a nucleic acid adjuvant.

According to some embodiments of the invention, the immune adjuvant comprises a nucleic acid adjuvant.

According to some embodiments of the invention, the nucleic acid adjuvant comprises a CpG oligodeoxynucleotide (CpG ODN).

According to some embodiments of the invention, the checkpoint inhibitor is administered prior to the UNO.

According to some embodiments of the invention, the checkpoint inhibitor is administered prior to the immune adjuvant.

According to some embodiments of the invention, the immune adjuvant is administered subsequent to the UNO.

According to some embodiments of the invention, the cancer is refractory to treatment with a PD-1 inhibitor.

According to some embodiments of the invention, the cancer is refractory to treatment with a PD-L1 inhibitor.

According to some embodiments of the invention, the cancer is refractory to treatment with a CTLA-4 inhibitor.

According to some embodiments of the invention, the cancer is refractory to treatment with a LAG-3 inhibitor.

According to some embodiments of the invention, UNO sensitizes the cancer to treatment with a PD-1 inhibitor by upregulating the expression of PD-1 or PD-L1 before the PD-1 inhibitor is administered.

According to some embodiments of the invention, UNO sensitizes the cancer to treatment with a PD-L1 inhibitor by upregulating the expression of PD-L1 before the PD-L1 inhibitor is administered.

According to some embodiments of the invention, UNO sensitizes the cancer to treatment with a CTLA-4 inhibitor by upregulating expression of CTLA-4 before the CTLA-4 inhibitor is administered.

According to some embodiments of the invention, the administration of UNO results in a higher tumor-specific immune cell response. In some embodiments, the higher tumor-specific immune cell response is an increase in tumor antigen-specific CD8+ T-cells.

According to some embodiments of the invention, the combination of UNO and a checkpoint inhibitor results in a higher tumor-specific immune cell response. According to some embodiments of the invention, the combination of UNO and a checkpoint inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a PD-1 inhibitor results in a higher tumor-specific immune cell response. In some embodiments, the higher tumor-specific immune cell response is an increase in tumor antigen-specific CD8+ T-cells.

According to some embodiments of the invention, the combination of UNO and a PD-1 inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a PD-L1 inhibitor results in a higher tumor-specific immune cell response. In some embodiments, the higher tumor-specific immune cell response is an increase in tumor antigen-specific CD8+ T-cells.

According to some embodiments of the invention, the combination of UNO and a PD-L1 inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a CTLA-4 inhibitor results in a higher tumor-specific immune cell response. In some embodiments, the higher tumor-specific immune cell response is an increase in tumor antigen-specific CD8+ T-cells. According to some embodiments of the invention, the combination of UNO and a CTLA-4 inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a LAG-3 inhibitor results in a higher tumor-specific immune cell response. In some embodiments, the higher tumor-specific immune cell response is an increase in tumor antigen-specific CD8+ T-cells. According to some embodiments of the invention, the combination of UNO and a LAG-3 inhibitor synergistically results in a higher tumor-specific immune cell response. In some embodiments, the higher tumor-specific immune cell response is an increase in tumor antigen-specific CD8+ T-cells.

According to some embodiments of the invention, the cancer is positive for the microsatellite instability (MSI) and/or the mismatch repair deficient (dMMR) marker.

According to some embodiments of the invention, the cancer is negative for the microsatellite instability (MSI) and/or the mismatch repair deficient (dMMR) marker.

According to some embodiments of the invention, the cancer is selected from the group consisting of colon, breast, melanoma (e.g., BRAF positive melanoma), lung (e.g., Non-Small Cell Lung Cancer (NSCLC)), Head and Neck Squamous Cell Cancer (HNSCC), Classical Hodgkin Lymphoma (cHL), Primary Mediastinal Large B-Cell Lymphoma (PMBCL), Urothelial Carcinoma, Gastric Cancer, Esophageal Cancer, Cervical Cancer, Hepatocellular Carcinoma (HCC), Merkel Cell Carcinoma (MCC), Renal Cell Carcinoma (RCC), Endometrial Carcinoma, Tumor Mutational Burden-High (TMB-H) Cancer, Cutaneous Squamous Cell Carcinoma (cSCC), Triple-Negative Breast Cancer (TNBC), Microsatellite Instability-Low, Microsatellite Instability-High, or Mismatch Repair Deficient Cancer, and Microsatellite Instability-Low, Microsatellite Instability-High, or Mismatch Repair Deficient Colorectal Cancer (CRC).

In some embodiments, UNO can be administered to treat solid tumors at a dose of one of 10,000 ppm, 15,000 ppm, 20,000 ppm, 25,000 ppm, 50,000 ppm, or 100,000 ppm through local administration, such as intra-tumoral injection. In some embodiments, the UNO can be administered for a duration of 5 minutes at a flow rate of 0.2 L/min. In some embodiments, the UNO can be administered for multiple cycles. In some embodiments, a cycle is a three-week period (21 days), wherein the UNO can be administered on day 1 and/or day 8 of one or more 21-day cycles. In some embodiments, a cycle is a four-week period (28 days), wherein the UNO can be administered on day 1 and/or day 8 and/or day 15 of one or more 28-day cycles. In some embodiments, the UNO can be administered at multiple tumor sites. In some embodiments, the UNO can be administered in combination with a checkpoint inhibitor, wherein the checkpoint inhibitor is one of a PD-1 inhibitor, PD-L1 inhibitor, a CTLA-4 inhibitor, and a LAG-3 inhibitor. In some embodiments, the checkpoint inhibitor can be administered intravenously subsequent to the administration of the UNO.

In some embodiments, UNO can be administered to treat primary or metastatic tumors at a dose of 50,000 ppm through local administration, such as intra-tumoral injection. In some embodiments, the UNO can be administered for a duration of 5 minutes at a flow rate of 0.2 L/min. In some embodiments, the UNO can be administered for multiple 21-day cycles, wherein the UNO can be administered on day 1 of one or more 21-day cycles.

In some embodiments, UNO can be administered to treat triple negative breast cancer (TNBC) at a dose of 50,000 ppm through local administration, such as intra-tumoral injection. In some embodiments, the UNO can be administered for a duration of 5 minutes at a flow rate of 0.2 L/min. In some embodiments, the UNO can be administered for multiple 21-day cycles, wherein the UNO can be administered on day 1 of one or more 21-day cycles. In some embodiments, the UNO can be administered in combination with a checkpoint inhibitor, wherein the checkpoint inhibitor is a PD-1 inhibitor. In some embodiments, the PD-1 inhibitor is pembrolizumab. In some embodiments, the pembrolizumab can be administered intravenously subsequent to the administration of the UNO. In some embodiments, the pembrolizumab is administered at a dose of 200 mg for a duration of 30 minutes every three weeks. In some embodiments, the pembrolizumab is administered at a dose of 400 mg for a duration of 30 minutes every six weeks.

In some embodiments, UNO can be administered to treat advanced cutaneous malignant melanoma at a dose of 25,000 ppm or 50,000 ppm through local administration, such as intra-tumoral injection. In some embodiments, the UNO can be administered for a duration of 5 minutes at a flow rate of 0.2 L/min. In some embodiments, the UNO can be administered for multiple 21-day cycles, wherein the UNO can be administered on day 1 of one or more 21-day cycles. In some embodiments, the UNO can be administered in combination with a checkpoint inhibitor, wherein the checkpoint inhibitor is a CTLA-4 inhibitor. In some embodiments, the CTLA-4 inhibitor is ipilimumab. In some embodiments, the ipilimumab is administered intravenously subsequent to the administration of the UNO, wherein the ipilimumab is administered at a dose of 3 mg/kg for a duration of 30 minutes. In some embodiments, the ipilimumab is administered for up to four cycles.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows a schematic representation of an experimental timeline for treating tumors in mice using UNO and a PD-1 inhibitor, pembrolizumab (Keytruda).

FIG. 2A-FIG. 2C show average primary tumor volumes at the indicated time points for the treatment depicted in FIG. 1. FIG. 2A and FIG. 2B show primary tumor volumes. Points represent mean±SD. In FIG. 2B, “X” indicates that a mouse was measured and sacrificed on the same day of measurement. FIG. 2C shows primary tumor volumes in mice with above median response. In particular, the figure shows the average tumor volume of the three best responding mice out of seven in each group (showing the highest reduction in tumor volume which was above the median of the group). FIG. 2C shows tumor volume over time, where each point in FIG. 2C represents mean±SD; the Y axis is a logarithmic scale; and p=˜0.1 on days 23-27 by one-way ANOVA unpaired t-test.

FIG. 3 shows average primary and secondary tumors volumes at the indicated time points for the treatment depicted in FIG. 1. Points represent mean±SD.

FIG. 4 shows average secondary tumor volumes at the indicated time points for the treatment depicted in FIG. 1. Points represent mean±SD. “X” indicates that a mouse was measured and sacrificed on the same day of measurement. *p<0.05, using Student's t-test.

FIG. 5 shows percentage of secondary tumor taken for the treatment depicted in FIG. 1. p=˜0.1 on Days 20-30.

FIG. 6A-FIG. 6C show percentages of complete and partial regression of primary and secondary tumors for the treatment depicted in FIG. 1. CT26 tumor-bearing mice were treated intratumorally with UNO or nitrogen (N₂) for 5 minutes, 8 days post primary tumor cell inoculation and three days post-secondary tumor cell inoculation. Pembrolizumab was administered i.p., two days prior to gas treatment, e.g., UNO or N₂, and continued for a total of 5 injections, every two days. FIG. 6A is a graph presenting the percentage of mice that showed complete tumor regression in each treatment relative to all mice that showed complete tumor regression. The treated primary tumor of four mice disappeared completely following intra-tumoral gas treatment. Specifically, three mice were treated with UNO for 5 minutes (¾=75%) and one mouse was treated with N₂ for 5 minutes (¼=25%). FIG. 6B is a graph presenting the percentage of responding mice [mice with complete primary tumor regression or partial tumor regression (primary tumor volume of mouse was reduced by at least 80% compared to the average tumor volume of untreated mice group)]21 days post primary tumor inoculation. Specifically, 5/7 mice in the UNO+pembrolizumab group responded (one showed complete tumor regression and four showed >80% reductions in primary tumor volume) while 2/6 mice in the N₂+pembrolizumab group responded, one showed complete tumor regression and one showed >80% reduction in primary tumor volume). FIG. 6C is a graph presenting percentage of partially responding mice (primary tumor volume of the mouse was reduced by at least 80% compared to the average tumor volume of the untreated mice group) 21 days post primary tumor inoculation. Specifically, 4/7 mice in the UNO+pembrolizumab group showed >80% reduction in the primary tumor volume compared to the average tumor volume of the untreated mice group, while 1/6 mice in the N₂+pembrolizumab group showed >80% reduction in the primary tumor volume compared to the average tumor volume of the untreated mice group.

FIG. 7 is a Kaplan-Meier graph demonstrating mice survival percentages up to 100 days following primary tumor inoculation for the treatment depicted in FIG. 1. *p=0.0848.

FIG. 8A-FIG. 8C shows the local effect of a treatment comprising UNO and a PD-1 inhibitor, e.g., anti-murine PD-1 (anti-mPD-1), on CT26 tumor-bearing mice. FIG. 8A shows the assay scheme for the treatment. FIG. 8B shows the tumor growth curves of CT26 tumor-bearing mice (average tumor volume on treatment day 71.91±37.24 mm³) treated with UNO for 5 or 10 minutes administered intratumorally by a 23 G hypodermic needle. Anti-mPD-1 dosing started 2 days before UNO treatment. 5-10 mg/kg of anti-mPD-1 was administered every 3 days, for a total of 4-5 times. Analysis via mixed model repeated measures (MMRM) with fixed effects for baseline tumor volume, study day, treatment by study day interaction, *p=0.0005 (at day 9 post UNO treatment). FIG. 8C shows the representative images of the primary tumor after treatment.

FIG. 9A-FIG. 9C show CT26 primary and secondary tumor-free mice after an exemplary treatment. FIG. 9A shows the assay scheme for the exemplary treatment. FIG. 9B shows the percentage of primary and secondary tumor-free mice 100 days post-UNO treatment. Statistical analysis: Fisher's Exact Test: P-value=0.1489, Pairwise Treatment Group Comparison—UNO ppm for 10 min+anti-mPD-1 vs. anti-mPD-1. FIG. 9C shows the representative images of 10 min of UNO+anti-mPD-1 treated mouse (left) versus control mouse (right).

FIG. 10A and FIG. 10B show the effect of UNO and anti-mPD-1 treatment on mice survival. FIG. 10A shows the assay scheme for the treatment. FIG. 10B shows the survival curve for the treatment, presented as a Kaplan Meier curve. P-value=0.065 for UNO+anti-mPD-1 vs. anti-mPD-1.

FIG. 11A and FIG. 11B show tumor progression after single UNO dosing (FIG. 11A) and repeated UNO dosing (FIG. 11B).

FIG. 12A and FIG. 12B show the average tumor volume 14 and 15 days after the first UNO treatment. FIG. 12A shows that the average volume of the tumors of 4T1 tumor-bearing mice treated with UNO and anti-mPD-1 was significantly smaller as compared to untreated tumors. Analysis via mixed model for repeated measures (MMRM) on day 15 after treatment with Kenward-Rodger's method, with ***P<0.001 ****P<0.0001. In addition, the primary tumors were 13.2% smaller following a single-dose of UNO alone compared to anti-mPD-1 alone. FIG. 12B shows that, 14 days after the first UNO treatment, the primary tumors of 4T1 tumor-bearing mice treated with repeated dosing of UNO with or without anti-mPD-1 were significantly smaller as compared to tumors that were untreated or treated with only anti-mPD-1. Tumors treated with repeated doses of UNO, without anti-mPD-1, were 21.0% smaller as compared to the anti-mPD-1 alone group. Analysis via mixed model for repeated measures (MMRM) on day 14 after treatment with Kenward-Rodger's method, with ****P<0.0001.

FIG. 13A and FIG. 13B show the prolonged survival of tumor-bearing mice following repeated doses of UNO without the resection of the primary tumor (FIG. 13A) and with primary tumor resection (FIG. 13B), as compared to anti-mPD-1 alone, untreated mice or N₂ controls. Kaplan Meier analysis for FIG. 13A: Comparison Hazard Ratio, UNO+anti-mPD-1 vs untreated: HR=0.39, p-value=0.0915, Log-Rank p-value=0.0649, [95% CI]=[0.13, 1.16]. Comparison Hazard Ratio, UNO+anti-mPD-1 vs anti-mPD-1: HR=0.51, p-value=0.2370, Log-Rank p-value=0.2149, [95% CI]=[0.17,1.50]. Comparison Hazard Ratio, UNO vs untreated: HR=0.63, p-value=0.3892, Log-Rank p-value=0.2585, [95% CI]=[0.22,1.79]. Comparison Hazard Ratio, UNO+anti-mPD-1 vs anti-mPD-1: HR=1.03, p-value=0.9581, Log-Rank p-value=0.9068, [95% CI]=[0.36,2.94]. Comparison Hazard Ratio, UNO+anti-mPD-1 vs UNO: HR=0.48, p-value=0.2075, Log-Rank p-value=0.2213, [95% CI]=[0.15,1.51]. Comparison Hazard Ratio, UNO+anti-mPD-1 vs N₂: HR=0.76, p-value=0.6108, Log-Rank p-value=0.6948, [95% CI]=[0.26,2.22]. Comparison Hazard Ratio, UNO+anti-mPD-1 vs N₂+anti-mPD-1: HR=0.76, p-value=0.6337, Log-Rank p-value=0.6073, [95% CI]=[0.25,2.32]. Kaplan Meier analysis for FIG. 13B: Comparison Hazard Ratio, UNO+anti-mPD-1 vs untreated: HR=0.35, p-value=0.057, Log-Rank p-value=0.0412, [95% CI]=[0.12,1.03]. Comparison Hazard Ratio, UNO+anti-mPD-1 vs anti-mPD-1: HR=0.35, p-value=0.0701, Log-Rank p-value=0.0723, [95% CI]=[0.11,1.09]. Comparison Hazard Ratio, UNO+anti-mPD-1 vs. UNO: HR=0.22, p-value=0.0465, Log Rank p-value=0.0475, [95% CI]=[0.05,0.98]. Comparison Hazard Ratio, UNO vs untreated: HR=0.69, p-value=0.4972, Log-Rank p-value=0.2585, [95% CI]=[0.24,2.01]. Comparison Hazard Ratio, UNO vs anti-mPD-1: HR=1.58, p-value=0.4412, Log-Rank p-value=0.7999, [95% CI]=[0.49,5.08]. Comparison Hazard Ratio, UNO+anti-mPD-1 vs N₂: HR=0.06, p-value=0.0085, Log-Rank p-value=0.0007, [95% CI]=[0.01,1.75]. Comparison Hazard Ratio, UNO+anti-mPD-1 vs N₂+anti-mPD-1: HR=0.65, p-value=0.3894, Log-Rank p-value=0.3894, [95% CI]=[0.24,1.75]. Hazard ratio and p-value were derived from Cox proportional hazard model.

FIG. 14A-FIG. 14C show the average tumor volumes and survivability for a treatment comprising UNO and a CTLA-4 inhibitor, e.g., anti-murine CTLA-4 (anti-mCTLA-4), on 4T1 tumor-bearing mice. FIG. 14A shows the growth of tumors treated with UNO, with and without anti-mCTLA-4, 13 days after a first UNO treatment (i.e., 10 days after the second treatment).

FIG. 14B shows that the average tumor volume of the tumors treated with UNO and anti-mCTLA-4 was significantly smaller as compared to tumors treated with anti-mCTLA-4 alone. N=8-10 mice per arm. Analysis via mixed model for repeated measures (MMRM) on day 13 after first UNO treatment (i.e., 10 days after the second treatment) with Kenward-Rodger's method, P=0.0029. The tumors of 4T1 tumor-bearing mice treated with only UNO were 12.9% smaller as compared to the tumors of the mice treated with only anti-mCTLA-4, P=0.1158. FIG. 14C shows the survival curve of 4T1 tumor-bearing mice treated with UNO twice, with and without anti-mCTLA-4. As depicted in the figure, UNO monotherapy significantly prolongs mice survival compared to anti-mCTLA-4 monotherapy. Comparison Hazard Ratio, UNO vs untreated: HR=0.31, p-value=0.0412, Log-Rank p-value=0.0313, [95% CI]=[0.10,0.95], UNO vs+anti-mCTLA-4: HR=0.16, p-value¹=0.027, Log-Rank p-value=0.0173, [95% CI]=[0.03,0.81], UNO+anti-mCTLA-4 vs untreated: HR=0.29, p-value=0.0720, Log-Rank p-value=0.0369, [95% CI]=[0.08,1.12], UNO+anti-mCTLA-4 vs anti-mCTLA-4: HR=0.42, p-value=0.2274, Log-Rank p-value=0.1839, [95% CI]=[0.10,1.72], UNO+anti-mCTLA-4 vs UNO: HR=0.61, p-value=0.4508, Log-Rank p-value=0.4809, [95% CI]=[0.17,2.22], UNO+anti-mCTLA-4 vs N₂: HR=0.66, p-value=0.4567, Log-Rank p-value=0.3659, [95% CI]=[0.22,1.99], UNO+anti-mCTLA-4 vs N₂+anti-mCTLA-4: HR=0.63, p-value=0.4924, Log-Rank p-value=0.5130, [95% CI]=[0.17,2.33]. Hazard ratio and p-value were derived from Cox proportional hazard model. 10 mice died at day 13 due to toxic reaction to an anti-mCTLA-4 antibody and were excluded from this analysis.

FIG. 15 shows average primary tumor volumes 14 days after treatments comprising UNO, anti-mPD-1, and/or a CpG ODN, e.g., a class B CpG ODN (CpG-B) on CT26 tumor-bearing mice. *p<0.05 vs. untreated mice, using one-way Anova-Tukey's test.

FIG. 16 shows the percentages of mice with complete tumor regression 42 days after the treatments described in FIG. 15. **p<0.01 UNO+anti-mPD-1+CpG-B treatment vs. untreated; *p<0.05 UNO+anti-mPD-1+CpG-B vs. UNO alone; p=0.117 UNO+anti-mPD-1+CpG-B vs. UNO+anti-mPD-1, using chi-square.

FIG. 17 shows the percentages of mice with distant tumor rejection 42 days after the treatments described in FIG. 15. On day 0, UNO+anti-mPD-1+CpG-B, with n=9, and in each of the other groups, n=10. 7 days following treatments, mice were sacrificed due to ethics limits. On day 42: untreated group, n=2 mice; UNO+anti-mPD-1 group, n=4 mice; UNO only group, n=2 mice; UNO+anti-mPD-1+CpG-B, n=6 mice.

FIG. 18 shows average distant tumor volumes 7 days after the treatments described in FIG. 15.

FIG. 19 is a Kaplan-Meier graph demonstrating mice survival percentages for the treatments described in FIG. 15 52 days after primary tumor inoculation. p<0.05 UNO+anti-mPD-1+CpG-B vs. UNO treatment on days 31-52; p<0.05 UNO+anti-mPD-1+CpG-B vs. untreated on days 40 and 42; p<0.01 UNO+anti-mPD-1+CpG-B vs. untreated on days 45 and 52; p=0.117 UNO+anti-mPD-1+CpG-B vs. UNO+anti-mPD-1 on days 45 and 52, using Chi-square.

FIG. 20 shows a schematic representation of the experimental timeline for the treatments described in FIG. 15.

FIG. 21 is a graph demonstrating the percentages of viable, early apoptotic, late apoptotic or necrotic CT26 cells following exposure to UNO. CT26 cells were exposed to UNO at 25,000 ppm, 50,000 ppm or 100,000 ppm for 10 seconds, 30 seconds or 1 minute, as indicated. The cell death type was analyzed 24 hours following UNO treatment using Annexin V/Propidium Iodide (PI) staining.

FIG. 22 is a graph demonstrating PD-L1 expression on CT26 PI negative cells following exposure to UNO. CT26 cells were exposed to UNO at 25,000 ppm, 50,000 ppm or 100,000 ppm for 10 seconds, 30 seconds or 1 minute, as indicated. The level of PD-L1 expression on the cells was assessed by a flow cytometry analysis, using labeled anti-PD-L1 antibodies.

FIG. 23 is a graph demonstrating mean fluorescence intensity (MFI) values of PD-L1 expression on CT26 PI negative cells following exposure to UNO. CT26 cells were exposed to UNO at 25,000 ppm, 50,000 ppm or 100,000 ppm for 10 seconds, 30 seconds or 1 minute, as indicated. The mean fluorescent intensity of PD-L1 expression on the cells was assessed by a flow cytometry analysis, using labeled anti-PD-L1 antibodies.

FIG. 24 shows the effects of UNO and anti-mCTLA-4 on systemic levels of tumor antigen-specific CD8+ T-cells 7 days after treatment.

### DESCRIPTION OF SPECIFIC EMBODIMENTS OF THE INVENTION

The present invention, in some embodiments thereof, relates to cancer treatment using UNO and a checkpoint inhibitor. In some embodiments, the treatment further includes the use of an immune adjuvant. In some embodiments thereof, the invention relates to UNO as a sensitizing treatment to checkpoint inhibitors.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cancer immunotherapy, including e.g., cell-based therapy, antibody therapy, cytokine or adjuvant therapy and vaccines, has emerged in the last couple of years as a promising strategy for treating various types of cancer. In parallel, tumor ablation is a minimally invasive technique that is commonly used in the treatment of solid tumors. In situ tumor ablation via delivery of an exogenous UNO to the solid tumor has been recently described.

While reducing specific embodiments of the present invention to practice, the present inventors have now discovered that treatment with a combination of UNO and a checkpoint inhibitor has a combined improved anti-cancer effect (Examples 1-2 of the Examples section which follows).

The present inventors have also discovered that treatment with a combination of UNO, a checkpoint inhibitor, and an immune adjuvant has a combined improved anti-cancer effect (Examples 4-6 of the Examples section which follows).

Consequently, the present teachings suggest the use of a combination of UNO, the checkpoint inhibitor and, optionally, an immune adjuvant for the treatment of cancer.

In some embodiments, the present teachings suggest the use of UNO as a sensitizing treatment for checkpoint inhibitors in cancer treatment, wherein the administration of UNO upregulates the expression of one or more immune checkpoint proteins.

Thus, according to an aspect of the invention there is provided a method of treating cancer in a subject in need thereof, the method comprising administering to the subject a therapeutically effective amount of UNO and a checkpoint inhibitor, thereby treating the cancer in the subject. According to some embodiments, the method further comprises administering an immune adjuvant. According to some embodiments, the UNO is administered prior to said checkpoint inhibitor.

According to an additional or an alternative aspect of the present invention, there is provided a combination of UNO and a checkpoint inhibitor, for use in treating cancer in a subject in need thereof. According to some embodiments, the combination further comprises an immune adjuvant. According to some embodiments, the UNO is provided to the subject prior to the checkpoint inhibitor.

As used herein the term “treating” refers to curing, reversing, attenuating, alleviating, minimizing, suppressing or halting the deleterious effects of a disease or disorder (e.g., cancer). Those of skill in the art will understand that various methodologies and assays can be used to assess the development of a pathology, and similarly, various methodologies and assays may be used to assess the reduction, remission or regression of a pathology (e.g., a malignancy), as discussed below.

As used herein throughout, the terms “subject” and “patient” are used interchangeably herein and refer to both human and nonhuman organisms, i.e., animals. The term “nonhuman animals” of the disclosure includes all vertebrates, e.g., mammals and non-mammals, such as nonhuman primates, sheep, dog, cat, horse, cow, chickens, amphibians, reptiles, and the like, for medical and/or laboratory research purposes. Preferably, the subject is a human subject. More preferably, the subject is a human patient diagnosed with cancer (e.g., pre-malignant or malignant tumor).

As used herein throughout, the term and “tumor” describes a plurality of cells, or a tissue composed of the plurality of cells that are characterized by abnormal cell growth and which serve no physiological function.

By “abnormal cell growth” it is meant uncontrolled, progressive proliferation of the cells, which is no longer under normal bodily control. The growth of a tumor tissue typically exceeds, and is uncoordinated with, that of the normal cells or tissues around it.

“Abnormal cell growth” also describes cell growth that is independent of normal regulatory mechanisms (e.g., loss of contact inhibition), including, for example, abnormal growth of: (1) cancerous (or cancer) cells that proliferate by expressing a mutated tyrosine kinase or over-expression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/threonine kinase activation occurs.

A tumor as described herein can be a primary tumor or a secondary tumor.

The term “malignant tumor” describes a tumor that is not self-limited in its growth, is capable of invading into adjacent tissues, and may be capable of spreading to distant tissues (metastasizing). The term “benign tumor” describes a tumor which is not malignant (i.e., does not grow in an unlimited, aggressive manner, does not invade surrounding tissues, and does not metastasize).

The term “primary tumor” describes a tumor that is at the original site where it first arose.

The term “secondary tumor” describes a tumor that has spread from its original (primary) site of growth to another site, close to or distant from the primary site, and is also referred to herein and in the art as metastasis, or as metastasizing tumor. The term “secondary tumor” as used herein also describes recurrent tumor, which can ne at the original site as the primary tumor and/or at another site, as a metastasizing tumor.

According to some of any of the embodiments described herein, the tumor is a malignant tumor, for example, a malignant cancerous tumor, and the tumor cells are cancer or cancerous cells.

According to these embodiments, the methods and uses as described herein in any of the respective embodiments are for treating cancer or a cancerous tumor is a subject in need thereof.

The methods and uses as described herein are in the context of subjects having a primary cancer tumor, a metastasizing cancer and/or a recurrent cancer, as described herein.

The term “cancer” encompasses malignant and benign tumors as well as disease conditions evolving from primary or secondary tumors, as described herein.

Examples of benign tumors include, without limitation, lipomas, chondromas, adenomas, pilomatricomas, teratomas, and hamartomas.

Cancers treatable according to embodiments of the invention include, but are not limited to, carcinomas, sarcomas, blastomas, and germ cell tumors. Carcinomas include, without limitation, adenocarcinomas (e.g., small cell lung cancer, kidney, uterus, prostate, bladder, ovary and/or colon adenocarcinoma) and epithelial carcinomas.

Examples of cancers treatable according to embodiments of the invention include, without limitation, adenocarcinoma, adrenal tumors (e.g., hereditary adrenocortical carcinoma), biliary tract tumors, bladder cancer, bone cancer, brain cancer, breast cancer (e.g., ductal breast cancer, invasive intraductal breast cancer, sporadic breast cancer, susceptibility to breast cancer, type 4 breast cancer, breast cancer-1, breast cancer-3, and/or breast-ovarian cancer), bronchogenic large cell carcinoma, cervical cancer (e.g., cervical carcinoma), carcinosarcoma, choriocarcinoma, cystadenocarcinoma, dermatofibrosarcoma protuberans, ductal carcinoma, Ehrlich-Lettre ascites, embryonal rhabdomyosarcoma, endocrine neoplasia, endometrial cancer (e.g., endometrial carcinoma), ependymoblastoma, epidermoid carcinoma, epithelial adult tumor, epithelioma, erythroleukemia (e.g., Friend and/or lymphoblast), extraskeletal myxoid chondrosarcoma, fibrosarcoma, gallbladder carcinoma, ganglioblastoma, gastrointestinal tract tumors (e.g., colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6, hereditary nonpolyposis type 7, small and/or large bowel carcinoma, esophageal carcinoma, tylosis with esophageal cancer, stomach carcinoma, pancreatic carcinoma, and/or pancreatic endocrine tumors), germ cell tumor (male germ cell tumor, and/or testicular and/or ovarian dysgerminoma), giant cell tumor, glial tumor, glioma, glioblastoma (e.g., glioblastoma multiforme, astrocytoma), head & neck cancer, heterohybridoma, heteromyeloma, histiocytoma, hybridoma (e.g., B-cell), hypernephroma, insulinoma, islet tumor, keratoma, large cell carcinoma, leiomyoblastoma, leiomyosarcoma, leukemia (e.g., acute lymphatic leukemia, acute lymphoblastic leukemia, acute lymphoblastic pre-B cell leukemia, acute lymphoblastic T cell leukemia, acute megakaryoblastic leukemia, monocytic leukemia, acute myelogenous leukemia, acute myeloid leukemia, acute myeloid leukemia with eosinophilia, B-cell leukemia, basophilic leukemia, chronic myeloid leukemia, chronic B-cell leukemia, eosinophilic leukemia, Friend leukemia, granulocytic or myelocytic leukemia, hairy cell leukemia, lymphocytic leukemia, mast cell leukemia, megakaryoblastic leukemia, monocytic leukemia, monocytic-macrophage leukemia, myeloblastic leukemia, myeloid leukemia, myelomonocytic leukemia, plasma cell leukemia, pre-B cell leukemia, promyelocytic leukemia, subacute leukemia, T-cell leukemia, lymphoid neoplasm, predisposition to myeloid malignancy, and/or acute nonlymphocytic leukemia), Li-Fraumeni syndrome, liposarcoma, liver cancer (e.g., hepatoblastoma, hepatocellular carcinoma, hepatocellular cancer, and/or hepatoma), lung cancer (e.g., Lewis lung carcinoma, small cell carcinoma and/or non-small cell carcinoma) lymphoma (e.g., Hodgkin's disease, non-Hodgkin's lymphoma, B-cell lymphoma, diffuse large B-cell lymphoma (DLBCL), Burkitt lymphoma, cutaneous T-cell lymphoma, histiocytic lymphoma, lymphoblastic lymphoma, T-cell lymphoma, and/or thymic lymphoma), lymphosarcoma, lynch cancer family syndrome II, mammary tumor, mastocytoma, medulloblastoma, medullary carcinoma, melanoma, mesothelioma, metastatic tumor, monocyte tumor, mucoepidermoid carcinoma, multiple glomus tumors, multiple meningioma, myelodysplastic syndrome, myeloma (e.g., multiple myeloma), nasopharyngeal cancer, nephroblastoma, nervous tissue glial tumor, nervous tissue neuronal tumor, neurinoma, neuroblastoma, neurogenic tumor, non-melanoma skin cancer, oat cell carcinoma, oligodendroglioma, osteochondroma, osteomyeloma, ovarian cancer (e.g., epithelial ovarian cancer, ovarian carcinoma, serous ovarian cancer, and/or ovarian sex cord tumors), papillary carcinoma, papilloma, paraganglioma (e.g., familial nonchromaffin), pheochromocytoma, pituitary tumor (invasive), placental site trophoblastic tumor, plasmacytoma, prostate cancer (e.g., prostate adenocarcinoma), renal cancer (e.g., Wilms' tumor type 2 or type 1), retinoblastoma, rhabdoid tumors (e.g., rhabdoid predisposition syndrome), rhabdomyosarcoma, sacrococcygeal tumor, sarcoma (e.g., Ewing's sarcoma, histiocytic cell sarcoma, Jensen sarcoma, myxosarcoma, osteosarcoma, reticulum cell sarcoma, soft tissue sarcoma and/or synovial sarcoma), schwannoma, small cell carcinoma, spindle cell carcinoma, spinocellular carcinoma, squamous cell carcinoma (e.g., in head and neck), subcutaneous tumor, teratocarcinoma (e.g., pluripotent), teratoma (e.g., immature teratoma of ovary), testicular cancer (e.g., testicular germ cell tumor), transitional cell carcinoma, Turcot syndrome with glioblastoma, thymoma, thyroid cancer (e.g., follicular, medullary and/or papillary thyroid cancer), trichoepithelioma, trophoblastic tumor, undifferentiated carcinoma, uterine cancer, uterine cervix carcinoma.

Methods and uses of the present embodiments can be used to treat one or more solid tumors.

As used herein, the term “solid tumor” refers to those conditions, such as cancer, that form an abnormal tumor mass, such as sarcomas, carcinomas, and lymphomas. For example, solid tumors can include, but are not limited to, ovarian tumors, prostate tumors, skin tumors, lung tumors, breast tumors, liver tumors, brain tumors, CNS tumors, kidney tumors, colon tumors, bladder tumors, intestinal tumors, melanomas, gliomas, ependymomas, oligodendrogliomas, oligoastrocytomas, astrocytomas, glioblastomas, and medulloblastomas. Suitable examples of solid tumor diseases include, but are not limited to, non-small cell lung cancer (NSCLC), neuroendocrine tumors, thyomas, fibrous tumors, metastatic colorectal cancer (mCRC), and the like. In certain embodiments, the solid tumor disease is an adenocarcinoma, squamous cell carcinoma, large cell carcinoma, and the like.

According to some embodiments, the cancer is or comprises a solid tumor, and can be, for example, adenocarcinoma, adrenal tumors (e.g., hereditary adrenocortical carcinoma), biliary tract tumors, bladder cancer, bone cancer, brain cancer, breast cancer (e.g., ductal breast cancer, invasive intraductal breast cancer, sporadic breast cancer, susceptibility to breast cancer, type 4 breast cancer, breast cancer-1, breast cancer-3, and/or breast-ovarian cancer), bronchogenic large cell carcinoma, cervical cancer (e.g., cervical carcinoma), carcinosarcoma, choriocarcinoma, cystadenocarcinoma, dermatofibrosarcoma protuberans, ductal carcinoma, Ehrlich-Lettre ascites, embryonal rhabdomyosarcoma, endocrine neoplasia, endometrial cancer (e.g., endometrial carcinoma), ependymoblastoma, epidermoid carcinoma, epithelial adult tumor, epithelioma, extraskeletal myxoid chondrosarcoma, fibrosarcoma, gallbladder carcinoma, ganglioblastoma, gastrointestinal tract tumors (e.g., colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6, hereditary nonpolyposis type 7, small and/or large bowel carcinoma, esophageal carcinoma, tylosis with esophageal cancer, stomach carcinoma, pancreatic carcinoma, and/or pancreatic endocrine tumors), germ cell tumor (male germ cell tumor, and/or testicular and/or ovarian dysgerminoma), giant cell tumor, glial tumor, glioma, glioblastoma (e.g., glioblastoma multiforme, astrocytoma), head & neck cancer, heterohybridoma, heteromyeloma, histiocytoma, hybridoma (e.g., B-cell), hypernephroma, insulinoma, islet tumor, keratoma, large cell carcinoma, leiomyoblastoma, liposarcoma, liver cancer (e.g., hepatoblastoma, hepatocellular carcinoma, hepatocellular cancer, and/or hepatoma), lung cancer (e.g., Lewis lung carcinoma, small cell carcinoma and/or non-small cell carcinoma) lymphoma (e.g., Hodgkin's disease, non-Hodgkin's lymphoma, B-cell lymphoma, diffuse large B-cell lymphoma (DLBCL), Burkitt lymphoma, cutaneous T-cell lymphoma, histiocytic lymphoma, lymphoblastic lymphoma, T-cell lymphoma, and/or thymic lymphoma), lymphosarcoma, lynch cancer family syndrome II, mammary tumor, mastocytoma, medulloblastoma, medullary carcinoma, melanoma, mesothelioma, metastatic tumor, monocyte tumor, mucoepidermoid carcinoma, multiple glomus tumors, multiple meningioma, myelodysplastic syndrome, myeloma (e.g., multiple myeloma), nasopharyngeal cancer, nephroblastoma, nervous tissue glial tumor, nervous tissue neuronal tumor, neurinoma, neuroblastoma, neurogenic tumor, non-melanoma skin cancer, oat cell carcinoma, oligodendroglioma, osteochondroma, osteomyeloma, ovarian cancer (e.g., epithelial ovarian cancer, ovarian carcinoma, serous ovarian cancer, and/or ovarian sex cord tumors), papillary carcinoma, papilloma, paraganglioma (e.g., familial nonchromaffin), pheochromocytoma, pituitary tumor (invasive), placental site trophoblastic tumor, plasmacytoma, prostate cancer (e.g., prostate adenocarcinoma), renal cancer (e.g., Wilms' tumor type 2 or type 1), retinoblastoma, rhabdoid tumors (e.g., rhabdoid predisposition syndrome), rhabdomyosarcoma, sacrococcygeal tumor, sarcoma (e.g., Ewing's sarcoma, histiocytic cell sarcoma, Jensen sarcoma, myxosarcoma, osteosarcoma, reticulum cell sarcoma, soft tissue sarcoma and/or synovial sarcoma), schwannoma, small cell carcinoma, spindle cell carcinoma, spinocellular carcinoma, squamous cell carcinoma (e.g., in head and neck), subcutaneous tumor, teratocarcinoma (e.g., pluripotent), teratoma (e.g., immature teratoma of ovary), testicular cancer (e.g., testicular germ cell tumor), transitional cell carcinoma, Turcot syndrome with glioblastoma, thymoma, thyroid cancer (e.g., follicular, medullary and/or papillary thyroid cancer), trichoepithelioma, trophoblastic tumor, undifferentiated carcinoma, uterine cancer, uterine cervix carcinoma.

According to a specific embodiment, the cancer is selected from the group consisting of colon, breast, melanoma, lung, Head and Neck Squamous Cell Cancer (HNSCC), Classical Hodgkin Lymphoma (cHL), Primary Mediastinal Large B-Cell Lymphoma (PMBCL), Urothelial Carcinoma, Gastric Cancer, Esophageal Cancer, Cervical Cancer, Hepatocellular Carcinoma (HCC), Merkel Cell Carcinoma (MCC), Renal Cell Carcinoma (RCC), Endometrial Carcinoma, Tumor Mutational Burden-High (TMB-H) Cancer, Cutaneous Squamous Cell Carcinoma (cSCC), Triple-Negative Breast Cancer (TNBC), Microsatellite Instability-High or Mismatch Repair Deficient Cancer and Microsatellite Instability-High or Mismatch Repair Deficient Colorectal Cancer (CRC).

According to a specific embodiment, the cancer is colon cancer.

According to a specific embodiment, the cancer is colon carcinoma.

According to a specific embodiment, the cancer is breast cancer.

According to a specific embodiment, the cancer is a respiratory tract tumor.

According to other specific embodiments, the cancer does not comprise a respiratory tract tumor.

According to specific embodiments, the cancer is refractory to treatment with a checkpoint inhibitor. This may present as innate resistance (also known as primary resistance, meaning that the patient does not respond at all to treatment) or an acquired resistance (also known as secondary resistance, meaning that patient initially responds to treatment but develops resistance later on).

As used herein the term “checkpoint inhibitor” refers to a molecule that inhibits the activity of one or more immune checkpoint proteins, resulting in activation of an immune cell.

As used herein the term “immune checkpoint protein” refers to an antigen independent protein that regulates an immune cell activation or function in response to an antigen. Immune checkpoint proteins can be either co-stimulatory proteins (i.e., transmitting a stimulatory signal resulting in activation of an immune cell) or inhibitory proteins (i.e., transmitting an inhibitory signal resulting in suppressing activity of an immune cell). According to some embodiments, the immune check-point protein regulates activation or function of a T cell. Numerous checkpoint proteins are known in the art and include, but not limited to, PD-1, PD-L1, A2aR, B7-H2, B7-H3, B7-H4, CTLA-4, CD73, CD80, CD86, LAG-3, TIM-3, NKG2A, PVRIG/PVRL2, CEACAM1, CEACAM 5/6, FAK, CCL2/CCR2, LIF, KIR, IDO, CD19, OX40, 4-1BB (CD137), CD27, CD47/SIRPα, CD70, CD40, CSF-1, GITR, CD28, IL-1, IL-1R3, IL-8, SEMA4D, Ang-2, CLEVER-1, Axl, phosphatidylserine, and ICOS (CD278).

According to specific embodiments, the immune checkpoint protein is presented on an immune cell e.g., T cell or antigen-presenting cells.

According to specific embodiments, the immune checkpoint protein is presented on a cancerous cell.

According to specific embodiments, cells of the cancer present said immune checkpoint protein or a binding pair thereof (i.e., receptor or ligand) following administration of said UNO.

Thus, according to specific embodiments, the method comprises determining presentation of an immune checkpoint protein or a binding pair thereof in a biological sample obtained from the subject. Methods of determining expression and/or presentation are well known in the art and include PCR, Western blot, immunostaining, flow cytometry, and the like.

According to specific embodiments, the immune checkpoint protein is selected from the group consisting of PD-1, PD-L1, CTLA-4, and LAG-3. According to specific embodiments, the immune checkpoint protein is PD-1. According to specific embodiments, the immune checkpoint protein is PD-L1. According to specific embodiments, the immune checkpoint protein is CTLA-4. According to specific embodiments, the immune checkpoint protein is LAG-3.

According to specific embodiments, the checkpoint inhibitor comprises an antibody.

Non-limiting examples of PD-L1 inhibitors include, without limitation, atezolizumab, durvalumab, avelumab, KN035, CK-301, AUNP12, CA-170 and BMS-986189; non-limiting examples of PD-1 inhibitors include, without limitation, pembrolizumab, nivolumab, cemiplimab, spartalizumab, sintilimab, tislelizumab, toripalimab, dostarlimab, JTX-4014, INCMGA00012, AMP-224, and AMP-514; non-limiting examples of CTLA-4 inhibitors include, without limitation, ipilimumab, tremelimumab; non-limiting examples of LAG-3 inhibitors include, without limitation, relatlimab.

According to specific embodiments, the cancer is refractory to treatment with a PD-1 inhibitor.

According to specific embodiments, the cancer is refractory to treatment with a PD-L1 inhibitor.

According to specific embodiments, the cancer is refractory to treatment with a CTLA-4 inhibitor.

According to specific embodiments, the cancer is refractory to treatment with a LAG-3 inhibitor.

As used herein, the term “immune adjuvant” refers to a substance that increases an antigen-specific activity of the immune response. As used herein “an adjuvant” does not refer to a modulator of an immune checkpoint molecule as described herein. Non-limiting examples of immune adjuvants that can be used with specific embodiments of the invention include mineral salt, aluminum salt, AS04, MF59, ASO1_{B}, an organic adjuvant, emulsion (e.g., Freund's Complete Adjuvant (FCA), Freund's Incomplete Adjuvant (FIA), Ribi Adjuvant System), microparticle, liposome, saponin, cytokine, microbial component and a nucleic acid adjuvant.

According to specific embodiments, the immune adjuvant is a nucleic acid adjuvant (e.g., a DNA molecule, an RNA molecule, a cDNA molecule). Such nucleic acid adjuvants are well known in the art and disclosed e.g., in Tmizoz et al. Current Opinion in Pharmacology (2018) 41: 104-113, the contents of which are fully incorporated herein by reference.

Non-limiting examples of nucleic acid adjuvants that can be used with specific embodiments of the invention include a cytosine-phosphorothioate-guanine oligodeoxynucleotide (CpG ODN), an expression vector of a cytokine, a microbial nucleic acid sequence, a viral nucleic acid sequence [e.g., double stranded RNA (dsRNA) of viral origin, virus-derived single stranded RNA (ssRNA)], polyinosinic-polycytidylic acid (Poly I:C), PIKA, imiquimod, resiquimod, cyclic dinucleotides (CDNs).

According to specific embodiments, the immune adjuvant is a CpG ODN.

CpG ODNs are short single-stranded synthetic DNA molecules that contain a cytosine nucleotide (“C”) followed by a guanine nucleotide (“G”) motif. The “p” refers to the phosphodiester link between consecutive nucleotides, although some ODN have a modified phosphorothioate (PS) backbone instead. When these CpG motifs are unmethylated, they act as immune adjuvants. Such CpG ODNs can be easily designed by the skilled in the art or commercially obtained from e.g., IDT, InvivoGen, Novus Biologicals, Creative Biogene and others.

According to specific embodiments, the cancer is positive for the microsatellite instability (MSI) [e.g., high microsatellite instability (MSI-H)] and/or the mismatch repair deficient (dMMR) marker.

According to other specific embodiments, the cancer is negative for the microsatellite instability (MSI) and/or the mismatch repair deficient (dMMR) marker.

Methods of determining MSI/dMMR are well known in the art and include Next-generation sequencing (NGS), Fluorescent multiplex PCR and C, immunohistochemistry, single-molecule molecular inversion probes (smMIPs).

One of skill in the art will appreciate that the methods and uses provided herein for inhibiting abnormal growth of tumor cells or tissue and are for treating cancer may be generally applicable to all known or to-be-discovered cancerous cell phenotypes and cancerous growths.

According to specific embodiments, cells of the cancer present PD-L1 and/or CTLA-4 and/or LAG-3 on their cell membrane.

Thus, according to specific embodiments, the method comprises determining presentation of one of PD-L1, CTLA-4, and/or LAG-3 in a biological sample obtained from the subject. Methods of determining expression and/or presentation are well known in the art and include PCR, Western blot, immunostaining, flow cytometry and the like.

The present embodiments relate to any size and shape of tumors, including large, spread and amorphic cancerous outgrowths.

The methods and uses provided herein may be especially useful for the treatment, control, and/or prevention of tumors (e.g., cancerous tumors) at localized sites, including inoperable tumors, tumors where localized treatment would be beneficial, and solid tumors.

According to some of any of the embodiments described herein, the methods and uses of the present embodiments are for inhibiting growth of cells of a primary tumor.

The present invention provides methods to treat cancer by a combined treatment comprising administration of UNO, preferably under a high pressure and for a sufficient time period, as described in further detail hereinunder.

According to a specific embodiment, an efficacious treatment with UNO is considered when between about 10% and about 100% (e.g., 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%) of the cancerous cells may be killed by the gaseous nitric oxide over the course of one or more administrations, as described herein in any of the respective embodiments.

UNO is defined as the delivery of gaseous nitric oxide in a preferably inert gas such as N₂ at a concentration of between about 10,000 and 1,000,000 ppm, as is described in further detail hereinafter.

The methods of delivering UNO may include administration of UNO in a continuous or pulsed manner.

According to some embodiments of the present invention, the methods are affected by locally administering the UNO.

By “locally administering of UNO” it is meant directly contacting the tumor cells or tissue with UNO, such that UNO is applied directly to the tumor and/or its close vicinity. In some embodiments, local administration of UNO is affected intratumorally, by applying UNO directly into the tumor cells or tissue. In some embodiments, local administration (intra-tumoral) of UNO is affected by applying UNO to the surface of the tumor tissue, for example, by contacting the surface of the tumor with UNO. In some embodiments, local administration is affected by bringing UNO in close vicinity to the tumor cells or tissue, for example, directly or up to 2 cm, or up to 1 cm, from at least one and preferably all of the tumor surfaces.

According to some of any of the embodiments described herein, the UNO is locally administered to the primary tumor and/or to a metastasizing tumor.

Exemplary organs to which UNO can be locally administered according to some of any of the respective embodiments as described herein include, but are not limited to, the adrenal gland, bladder, bones, brain, breast, cervix, colon, colorectum, esophagus, gastrointestinal tract, heart, kidney, liver, large intestine, lungs, mouth, ovaries, pancreas, parathyroid, pituitary gland, prostate, salivary gland, skin, small intestine, spleen, stomach, thymus, thyroid, testicles, urinary tract, uterus, or vagina. According to some embodiments, the UNO is locally administered to the liver, in case of a primary liver cancer or of liver metastases.

According to some embodiments, the UNO is locally administered to the colon, e.g., in case of a primary colon cancer or of colon metastases.

In some of any of the embodiments described herein, local administration is affected intratumorally, such that UNO is injected or otherwise delivered into the tumor.

In some of any of the embodiments described herein, local administration is affected by spraying or otherwise applying UNO onto at least one of the tumor's surfaces. At least some of the UNO then enters into the tumor via, e.g., diffusion.

In some of any of the embodiments described herein, local administration is affected by exposing the tumor to UNO in a close container, such that UNO is contacted with the tumor and enters into the tumor via e.g., diffusion. The container can be open or closed and can be sized to conform to the contours of the tumor.

In some of any of the embodiments described herein, local administration is affected by delivering UNO to a physiological space or cavity which contains at least a portion of the tumor tissue, such that the tumor is contacted with the UNO, and the UNO enters the tumor via e.g., diffusion.

According to some of any of the embodiments described herein for local administration of UNO is administered locally, as described herein.

In some of any of the embodiments described herein, the high dose of UNO is reflected by its relatively high concentration in the total amount of gas that is locally administered to the tumor and is presented by ppm (part per million) units.

In some of any of the embodiments described herein, the high dose of UNO is presented as its fraction, in ppm units, in the gas carrier. The gas carrier can be air, and preferably an inert gas such as N₂ or argon (Ar), preferably N₂.

In some of any of the embodiments described herein, the high dose of UNO is reflected by the mass of UNO that is locally administered to the tumor, per a volumetric unit of the tumor.

According to some of any of the embodiments described herein, the UNO is administered, as described herein in any of the respective embodiments, at a concentration of from about 10,000 ppm to about 1,000,000 ppm (1% to 100%), including any intermediate values and subranges therebetween, for example, from about 10,000 ppm to about 200,000 ppm, or from about 10,000 ppm to about 100,000 ppm, or from about 15,000 ppm to about 100,000 ppm, or from about 20,000 ppm to about 100,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and subranges between any of the foregoing, or is about 50,000 ppm.

In some of any of the embodiments described herein in the context of high dose of UNO, the high dose can be obtained by locally administering UNO at a dose of at least 10,000 ppm, or at least 20,000 ppm, or at least 50,000 ppm, and optionally up to about 1,000,000 ppm.

In exemplary embodiments, the UNO is administered, as described herein in any of the respective embodiments, at a concentration of about 20,000 to 200,000 ppm.

In exemplary embodiments, the UNO is administered, as described herein in any of the respective embodiments, at a concentration of about 20,000 to 100,000 ppm.

In exemplary embodiments, the UNO is administered, as described herein in any of the respective embodiments, in a concentration of about 50,000 ppm.

In exemplary embodiments, the UNO is administered, as described herein in any of the respective embodiments, at a concentration of about 25,000 ppm.

In exemplary embodiments, the UNO is administered, as described herein in any of the respective embodiments, at a concentration of about 20,000 ppm.

In exemplary embodiments, the UNO is administered, as described herein in any of the respective embodiments, at a concentration of about 10,000 ppm.

In exemplary embodiments, the UNO is administered, as described herein in any of the respective embodiments, at a concentration of about 100,000 ppm.

In exemplary embodiments, the UNO is administered, as described herein in any of the respective embodiments, at a concentration of from about 100,000 ppm to about 200,000 ppm, or from about 200,000 ppm to about 500,000 ppm, or from about 500,000 ppm to about 1,000,000 ppm or from about 50,000 ppm or about 100,000 ppm or about 200,000 ppm or about 500,000 ppm or about 1,000,000 ppm.

According to some of any of the embodiments described herein, the UNO is administered, as described herein in any of the respective embodiments, at a volumetric flow rate of from about 0.00001 LPM to about 10 LPM, preferably from about 0.0001 LPM and about 1 LPM, or from about 0.001 LPM and 0.5 LPM, including any intermediate values and subranges therebetween. For example, the volumetric flow rate can be from about 0.001 LPM to about 0.01 LPM, or from about 0.01 LPM to about 0.1 LPM, or from about 0.1 LPM to about 0.25 LPM, or from about 0.25 LPM to about 0.5 LPM, or from about 0.5 LPM to about 1 LPM, or from about 1 LPM to about 2 LPM, or from about 2 LPM to about 3 LPM, or from about 3 LPM to about 4 LPM, or from about 4 LPM to about 5 LPM, or from about 5 LPM to about 6 LPM, or from about 7 LPM to about 8 LPM, or from about 8 LPM to about 9 LPM, or from about 9 LPM to about 10 LPM, including any intermediate values and subranges therebetween, or it can be, for example, about 0.0001 LPM, or about 0.001 LPM, or about 0.01 LPM, or about 0.1 LPM, or about 1 LPM or about 10 LPM.

According to some of any of the embodiments described herein, the UNO is administered, as described herein in any of the respective embodiments, at a volumetric flow rate of from about 0.001 LPM to about 0.5 LPM.

According to some of any of the embodiments described herein, the UNO is administered, as described herein in any of the respective embodiments, at a volumetric flow rate of about 0.2 LPM.

According to some of any of the embodiments described herein, the UNO is administered for a time period that ranges from about 0.1 seconds to about 10 hours, per administration, including any intermediate values and subranges therebetween. For example, the time period can be from about 0.1 second to about 1 hour, or from about 1 second to about 10 minutes, or from about 1 minute to about 10 minutes, or from about 10 seconds to about 10 minutes, or from about 0.1 second to about 10 minutes, or from about 30 seconds to about 3 minutes, or from about 1 minute to about 30 minutes, or from about 10 minutes to about 60 minutes, or from about 60 minutes to about 180 minutes, or from about 180 minutes to about 600 minutes, including any intermediate values and subranges between any of foregoing, or it can be about 30 seconds, about 10 minutes, about 30 minutes, or about 60 minutes.

According to some of any of the embodiments described herein, the UNO is administered for a time period that ranges from about 30 seconds to about 10 minutes.

According to some of any of the embodiments described herein, the UNO is administered for about 5 minutes.

According to some of any of the embodiments described herein, the UNO is administered intermittently, i.e., more than once per day; e.g., such that a time of administration per day according to any of the respective embodiments described herein represents a sum of two or more separate administration periods, which may be of the same length or of different lengths.

In some of any of the embodiments, UNO can be administered, as described herein in any of the respective embodiments, at a dose of at least 10,000 ppm, and optionally up to about 1,000,000, or up to about 500,000 ppm, or up to about 200,000 ppm, or up to about 100,000 ppm, for a time period of from about 1 second to about 60 minutes at a volumetric flow (flow volume) of from about 0.0001 liter per minute (LPM) to about 1 LPM, including any intermediate values and subranges between any of the foregoing.

In some of any of the embodiments, UNO can be administered at a dose of at least 10,000 ppm, or at least 20,000 ppm, or at least 50,000 ppm, and optionally up to about 1,000,000 ppm, for a time period of at least 1 second, or at least 10 seconds, or at least 30 seconds, or at least 1 minute, and optionally up to about 60 minutes, at a volumetric flow rate (flow volume) of at least 0.0001 liter per minute (LPM), or at least 0.001 LPM, or at least 0.01 LPM, and optionally up to about 1 LPM, including any intermediate values and subranges between any of the foregoing.

Alternatively, or in addition, the amount of UNO administered to the tumor ranges from about 0.1 mg to about 300 mg, per cm³ tumor, per administration.

The parameters of the UNO concentration (ppm), volumetric flow rate (LPM) and time to achieve a desired mass of UNO, and vice versa, the UNO mass achieved by administering UNO at a concentration, volumetric flow and time, can be calculated using the known ideal gas equation, PV=nRT, wherein P is the pressure, V is the volume, n is the number of moles, R is the gas constant and T is the temperature.

More specifically, these relationships can be calculated or converted one to the other using the following equations:

$$1 ) X = y \times 1 {0}^{- 6}$$

$$2 ) V = \dot{V} \times t$$

$$3 ) n = \frac{V \times 1 {0}^{- 3} \times 101325}{8.314 \times 298}$$

$$4 ) m = n \times X \times 30.01 \times 1 {0}^{3}$$

$$5 ) m = ( \frac{( \dot{V} \times t ) \times 1 {0}^{- 3} \times 101325}{8.314 \times 298} ) \times ( y \times 1 {0}^{- 6} ) \times 30.01 \times 1 {0}^{3}$$

y is the concentration in ppm units;
X is the concentration in molar fraction units, and equation 1 presents the relation between molar fraction and ppm (y);
V is the volume, \dot{V} is the volumetric flow in LPM and t is the time in minutes, and equation 2 presents the relation between Volume, volumetric flow rate and time;
Equation 3 is the ideal gas equation, and the 10⁻³ factor is added for transformation from Liters to m³;
Equation 4 presents the relation between the mass (m) the mole number (n), the molar fraction X and Nitric Oxide molar mass (30.01 grams/mol); and
Equation 5 is a combination of equations 1-4 into a single equation, reflecting the relation between the mass of UNO and the molar fraction, volumetric flow and time. The 10³ is for obtaining the mass m on a milligram (mg) scale.

Thus, for example, at a volumetric flow of 0.1 LPM, time of 1 minute and concentration of 50,000 ppm, using equation 5 above, about 6.1-6.2 mg UNO is administered.

According to some of any of the embodiments described herein, the UNO is administered, as described herein in any of the respective embodiments, at a concentration of from about 10,000 ppm to about 1,000,000 ppm (1% to 100%), preferably from about 10,000 ppm to about 500,000 ppm, or from about 10,000 ppm to about 100,000 ppm, or at about 50,000 ppm; at a volumetric flow rate of from about 0.0001 LPM to about 10 LPM, preferably from about 0.001 LPM and 1 LPM; and during a time period that ranges from about 1 second to about 30 minutes, per administration.

According to some of any of the embodiments described herein, the UNO is administered, as described herein in any of the respective embodiments, at a concentration (dose) of from about 20,000 ppm to about 100,000 ppm or from about 20,000 ppm to about 50,000 ppm; for a time period that ranges from about 30 seconds to about 10 minutes; at a volumetric flow (flow volume) of from about 0.001 LPM to about 0.5 LPM, per administration.

According to some of any of the embodiments described herein, the UNO is administered, as described herein in any of the respective embodiments, in an amount of no more 1 mg UNO per 100 mm³ tumor volume, per administration, so as to avoid possible damage to healthy tissues adjacent to, or surrounding, the treated tumor.

According to some of any of the embodiments described herein, the UNO is administered, as described herein in any of the respective embodiments, in an amount of about 250 mg per cm³ tumor, per administration.

According to some of any of the embodiments described herein, the UNO is administered, as described herein in any of the respective embodiments, in an amount of from about 0.01 mg to about 100 mg, or from about 0.1 to about 10 mg per a tumor of 20 mm³ or less, including any intermediate values and subranges therebetween.

According to some of any of the embodiments described herein, the UNO is administered, as described herein in any of the respective embodiments, in an amount of from about 0.1 to about 300 mg including any intermediate values and subranges therebetween, per 1 cm³ tumor, per administration. For example, the UNO is administered in an amount of from about 0.1 mg to about 250 mg, or from 0.1 mg to about 100 mg, or from 1 mg to about 50 mg, or from about 1 mg to about 100 mg, or from about 1 mg to about 300 mg, of from about 50 mg to about 100 mg, or from about 50 mg to about 300 mg, or from about 100 mg to about 150 mg, or from about 100 mg to about 300 mg, or from about 10 mg to about 100 mg, or of from about 10 mg to about 250 mg, or from about 0.1 mg to about 10 mg, or from about 10 mg to 200 mg, including any intermediate values and subranges of any of the foregoing, per 1 cm³ tumor, per administration.

According to some of any of the embodiments described herein, the UNO is administered, as described herein in any of the respective embodiments, to a tumor having a volume of up to 20 mm³, and an amount of UNO that is administered as described herein in any of the respective embodiments is from about 0.001 mg to about 10 mg, or from about 0.01 mg to about 20 mg, or from about 0.01 mg to about 2 mg, or from about 0.1 mg to about 1.0 mg, or from about 0.2 mg to about 0.8 mg, including any intermediate values and subranges between any of the foregoing, per administration. In other embodiments, the tumor has a volume greater than 20 mm³.

Without being bound by any particular theory, it is assumed that a high dose (concentration or amount) as described herein in any of the respective embodiments, inhibits the growth of tumor cells, reduces tumor volume and/or stimulates an anti-tumor immune response, as described herein in any of the respective embodiments, without causing a harmful effect to healthy tissues in the vicinity of the tumor.

For any of the embodiments described herein for administration of UNO, the administration can be either continuous or pulsed, such that for each administration, the indicated dose of UNO is administered either continuously or in a pulsed manner. When the dose is referred to in ppm units, each pulse is at the indicated dose concentration, as described herein in any of the respective embodiments. When the dose is referred to as the total mass per administration, the indicated dose is divided into pulses.

According to some of any of the embodiments described herein, UNO is pulsed from about 2 to about 50 times, or from about 2 to about 30 times, or from about 2 to about 20 times, or from about 2 to about 15 times, or from about 5 to about 15 times, including any intermediate values and subranges therebetween, or about 10 times, per administration.

According to some of any of the embodiments described herein, each pulse is between 10,000 ppm and about 1,000,000 ppm of UNO, at a volumetric flow (flow volume) of from about 0.00001 LPM to about 0.5 LPM, wherein each pulse is, independently, between about 0.1 second and about 10 minutes per pulse with a break of from about 0.1 second to about 10 minutes between pulses.

According to some of any of the embodiments described herein, each pulse of UNO is, independently, from about 10 seconds per pulse to about 45 seconds per pulse, including any intermediate values and subranges therebetween.

According to some of any of the embodiments described herein, each pulse of UNO is about 30 seconds per pulse.

According to some of any of the embodiments described herein, UNO is not administered between pulses and the time between each two pulses is, independently, from about 1 second to about 300 seconds, or from about 1 second to about 200 seconds, or from about 1 second to about 100 seconds, or from about 1 second to about 50 seconds, or from about 10 seconds to about 50 seconds, including any intermediate values and subranges therebetween, or is about 20 seconds.

According to some of any of these embodiments, the ratio between the time of UNO pulsed administration and the resting time between pulses ranges from 1:2 to 1:5. For example, for each pulse of UNO administration for 5 seconds, a following resting time is independently from 10 to 50 seconds. Preferably, the UNO is pulsed such that about 33% of the time UNO is delivered and 66% of the time is resting or waiting time between pulses.

According to some of any of the embodiments described herein, the UNO is administered, as described herein in any of the respective embodiments, at two or more administration sites in or on the tumor (depending in the administration mode). In some of these embodiments, the distance between the two administration sites is, independently, from about 2.5 mm to about 1 cm, or from about 0.25 cm to about 0.5 cm, including any intermediate values and subranges therebetween.

When UNO is administered to two or more tumor sites, each administration is at the ppm dose or mass amount indicated herein in any of the respective embodiments, or the total mass (amount) administered to all tumor sites is as indicated herein in any of the respective embodiments.

According to some of any of the embodiments described herein, the method further comprises scavenging excess UNO from the one or more administration sites, as described in further detail hereinunder. In exemplary embodiments, the scavenging comprises applying a reduced pressure (vacuum) around the administration site(s).

According to some of any of the embodiments described herein for high dose administration of UNO, the administration is performed one or more times per a treatment session.

In some embodiments, it is performed once during a treatment session. In some embodiments, it is performed twice, thrice or more times during a treatment session. In some of these embodiments, the administration is performed once daily during the treatment session. Preferably, the administration is performed such that a time interval between the two administrations is at least one day, or at least two days, or at least three days, or at least four days, or at least five days, or at least six days, or at least one week, during a treatment session.

The duration of a treatment session can be determined by skilled persons such as physicians, in accordance with the subject's response to the treatment, that is, in accordance with the effect of the treatment on the growth of the cells of the primary and/or secondary tumor, as described herein.

According to some of any of the embodiments described herein for administration of UNO, the administration is performed once a day, although two or more times a day are also contemplated.

According to some embodiments, the local administration of UNO comprises injecting, or exposing the outer layer of a tumor to, or filling a space or cavity containing a tumor with, a high dose UNO, and in some embodiments, the UNO is at a concentration ranging from about 10,000 ppm to about 1,000,000 ppm, preferably 25,000, or 50,000 ppm, or 100,000 ppm, as described herein in any of the respective embodiments. The local administration to the tumor is for a period of time from about 1 second to about 3 hours, depending on the size and location of the tumor, with a very low volume in the order of up to 0.1 LPM and preferably 0.01 LPM.

According to some of any of the embodiments described herein, in cases where the tumor is covered by skin, peritoneum, crust or any other thick layer, this layer can be removed prior to or during exposure of the tumor to the UNO local administration.

In any of the embodiments described herein in the context of UNO administration, the UNO may be provided by an external source, for example, a reservoir of UNO or a chemical generator of UNO. In some embodiments, UNO is provided by a reservoir of UNO, preferably of a small volume of, for example, a single administration dosage (that is, the dose of UNO used per a single administration, as described herein in any of the respective embodiments). Such reservoirs are described in further detail hereinunder.

The UNO is preferably of medical purity, that is, preferably at least about 95%, more preferably at least about 99%, and even more preferably at least about 99.5% pure UNO. The UNO is preferably provided as a mixture of UNO and other gases, such as air, N₂, oxygen (O₂), and so forth, preferably an inert gas such as, for example, N₂, and its ppm concentration is within the gas it is mixed with.

Embodiments of the present invention further relate to a system, which is also referred to herein interchangeably as “device”, which is configured for locally administering UNO to a tumor as described herein in any of the respective embodiments. Such a system is also referred to herein as a delivery system. Such a system is also described in WO2021/105900, which is fully incorporated herein by reference.

Generally, but not obligatory, a system for locally administering UNO can include a pressure regulator, a flow meter, optionally an exposure box or container, one or more delivery lines, which are optionally terminated by or connected to a delivery device or configuration through which the UNO is administered and further optionally, a NO and/or NOx (as defined hereinunder) detector. Purging the UNO delivery system with an inert gas, such as N₂, may be desired.

The volume and/or flow rate of the administered (delivered) gas can be regulated by a digital flow controller, designed to deliver low volumes or flow rates of gas, of less than 0.1 LPM per cm³ of tissue, in accordance with any of the respective embodiments as described herein. Purging of the UNO delivery system, including purging the pressure regulator, flow meter and delivery lines can be performed before and/or after UNO local administration. The gas purge can preferably last at least 1 minute or until the NO and NOx (as described below) detectors read no signal. In exemplary embodiments, N₂ is used as the purging gas at a flow rate of at least 0.5 LPM.

According to some of any of the embodiments described herein, the delivery device is inserted into a body and advanced adjacent to an administration site, on or near tumor cells or tissue. When a delivery device or configuration is appropriately positioned, UNO is supplied and exits the device into the tumor or on or above a surface of the tumor, depending on the nature of the delivery device and the local administration mode of choice. In some embodiments, the delivery device extends against a tumor in order to form a seal, thus further reducing damage that may be caused by the UNO to adjacent normal cells that are outside of the area sealed off by the device.

The delivery device as described herein is meant to describe a component or configuration of the delivery system through which the gas exits the delivery system and contacts the administration site (e.g., the tumor or its close vicinity).

According to some of any of the embodiments described herein, administering UNO to a tumor as described herein in any of the respective embodiments can be accomplished by delivery device means such as one or more needles, including, for example, perforated needles, perforated spray needles, non-perforated and non-spray needles, umbrella needles, closed-tip needles, or other needles. The needles can optionally be nano-sized, micron-sized or macro-sized needles (having a diameter of 1 mm or higher). Other delivery devices are described hereinunder. Embodiments in which needles are used are typically used when the UNO is locally administered intratumorally, e.g., by intra-tumoral injection.

In some of any of the embodiments described herein in the context of a delivery system, the opening from which the UNO is delivered can be adjusted to the size of the tumor, so as to prevent damage to the area surrounding the cancer. In some embodiments, the opening does not exceed the size of the tumor.

The methods of locally administering UNO to a tumor can include contacting at least a portion of the tumor with the gaseous nitric oxide, and subsequently removing gaseous nitric oxide and NOx gas molecules from the treated site during or after the administration step. NOx encompasses NO, when x is 1, and oxidized forms of NO, which can be formed when UNO is in contact with a physiological environment and/or the subject's environment, whereby x can be, for example, 2. Vacuuming the gas can be done in a pulsed or continuous manner, preferably synchronized with the UNO mode of administration.

A delivery system can include a full-body, or a differently sized chemical hood designed to evacuate excessive UNO or NOx during treatment, in a pulsed or continuous manner, preferably synchronized with the UNO mode of administration.

The delivery system can include an evacuation cylinder, which is connected directly to a regulator of the UNO tank. To purge the regulator safely, the evacuation cylinder can be filled with a gas accumulated in the regulator.

Another embodiment of controlling UNO includes the use of a one-way valve where disconnecting the regulator or flow meter from the cylinder locks the valve, thereby preventing gas release from a gas tank.

According to some of any of the embodiments described herein, the methods, uses and delivery systems as described herein utilize a UNO cylinder, optionally equipped with a gas regulator, and one or more valves, and further optionally, the cylinder further comprises a delivery device for executing the local administration. The delivery device is in fluid communication with the cylinder, preferably via the valves and gas regulator (e.g., flow controller). Alternatively, the cylinder comprises means to connect the delivery device to the cylinder, to obtain fluid communication therebetween. The delivery device can be, for example, a scope with an annular-shape or a needle or a device configured for spraying the tumor, or else, as described in further detail hereinunder.

According to some of any of the embodiments described herein, the UNO cylinder is a miniature or at least portable cylinder.

According to some embodiments, the cylinder is of a volume of less than 1 liter, or less than 0.8 liter, or less than 0.75 liter, or less than 0.5 liter, or less than 0.3 liter.

The cylinder is preferably under low pressure, such as less than about 40 bar (about 600 psi). Thus, the cylinder can deliver about 10 liters of UNO. A regulator can optionally limit output pressure to about 50 psi, for example, and can be connected to an emergency on/off valve. The gas flow, in a case of, for example, a delivery system configured for injection, can be less than about 0.1 LPM or about 0.05 LPM. The use of such low flow rates during local administration of UNO can assist in limiting the exposure to the tumor or cancerous cells and protect the surrounding area, or healthy cells and tissues. In other embodiments, the cylinder can be under high pressure, such as greater than 40 bar, such as about 60 bar (about 870 psi), or about 80 bar (about 1160 psi), or about 100 bar (1450 psi). In other embodiments, the cylinder can be under a pressure higher than 100 bar.

According to some of any of the embodiments described herein, the methods and uses involve scavenging UNO and optionally other gases, and the scavenging can be performed by applying vacuum so as to remove UNO and other gases from the administration site. A delivery system as described herein is configured, according to some embodiments, as being capable of scavenging UNO from an administration site.

A delivery system according to some of the present embodiments can additionally or alternatively comprise one or more, preferably two, vacuum devices for scavenging gaseous nitric oxide and other gases that may form during the local administration. The vacuum devices can be placed or held above, or distal to, the tumor, or to the delivering device, during administration, for example, about 15 cm away. The vacuum devices can vacuum all the gases from the area at a rate of at least about 50 liters per minute. The vacuuming of the gas can be done in a pulsed or continuous manner, preferably synchronized with UNO administration. Purging the UNO delivery system, for example, with N₂, before and/or after can also be performed. Purging can also be performed intermittently during the procedure.

According to some embodiments, a chemical hood is placed above the tumor or tumor mass and used to apply vacuum and scavenge UNO. The hood can be placed about 15 cm above the tumor. The hood can vacuum gas at a flow rate of at least about 50 LPM.

A whole-body chemical hood that can accommodate the patient's body can also be used. The patient's head can be placed outside the hood to minimize the risk of breathing NOx molecules. UNO can be delivered as described herein. The vacuum system can exchange the gas at a flow rate of at least 50 liters per minute.

In each of the embodiments that relate to a vacuum application, a NO and NOx filtering pump can be placed in the discharge line. A soda lime filter, such as a Sofnolime filter, or a similar filter that can absorb NOx molecule, can be used. In each instance, vacuuming can be done in a pulsed or continuous manner, preferably synchronized with UNO administration or not.

According to some of any of the embodiments described herein, the delivery system is configured for delivering UNO to one or more administration sites by a positive pressure gradient, and scavenging UNO from the one or more administration sites by a negative pressure gradient. In this way, the delivery system may deliver UNO to one or more administration sites with reduced or nullified damage to collateral host cells.

It is to be noted that a certain level of damage to collateral cells may be tolerated, and that the conditions under which the UNO gas is administered may be optimized to decrease damage to collateral cells while also providing the therapeutic effects described herein.

According to some of any of the embodiments described herein, the delivery system comprises a gas supply passage in fluid communication with gas supply openings. The delivery system can further comprise an exhaust passage in fluid communication with an exhaust opening. The delivery system can comprise one or more gas supply openings and/or one or more exhaust openings, and/or one or more gas supply passages and/or one or more exhaust passages.

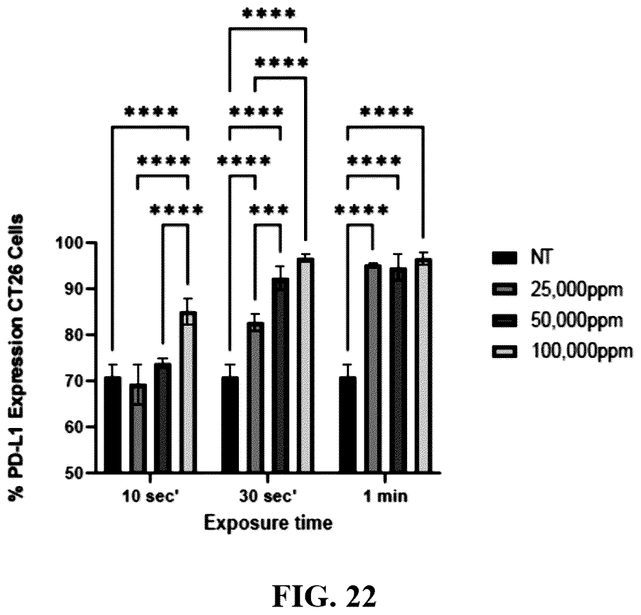

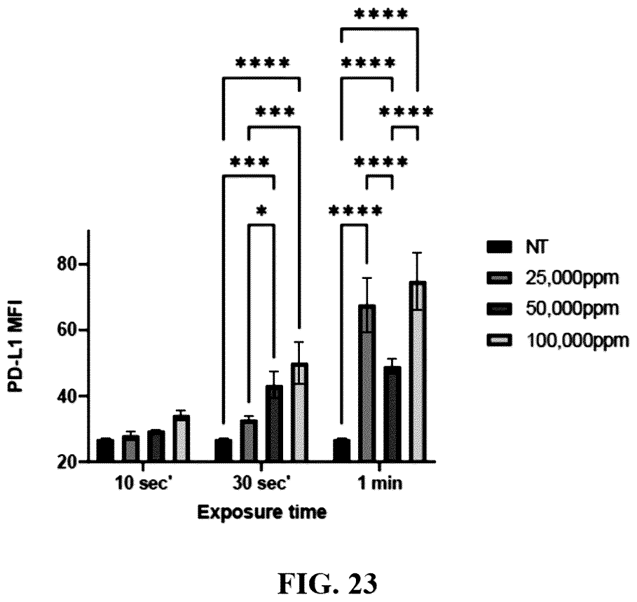

An exemplary system for the local administration and scavenging of gaseous nitric oxide comprises a container or box, as illustrated, for example in FIG. 22 of WO2021/105901 and described in further detail hereinunder, that can be filled with UNO at a volume of at least 0.5 Liter Per Minute (LPM) supplied from a tank (a gas reservoir). The container can have 2 or more holes, or ports. A first hole or port can be an input hole, sized to allow insertion of at least a portion of the tumor or of a bodily organ containing the tumor. An output hole can be connected to a discharge conduit or pipe that can remove or evacuate excessive NOx gases out from the box to the outside air, avoiding or minimizing the risk of contaminating the room and overexposing staff and the treated subjects (patients). Applying a vacuum can be in a pulsed or continuous manner, preferably synchronized with UNO administration.

An exemplary delivery system can comprise a small-bore inner cannula that delivers an adequate dose of UNO to the target site, for example, a target site of from about 1 mm² to about 2 cm² in size. The delivery system can further comprise an outer lumen through which a vacuum may be applied to scavenge excess UNO away from tumor cells and tissue that surround and border the tumor site. Such a configuration allows locally administering UNO to the target site (a tumor), without excessive damage to healthy host tissues.

In an exemplary method, a tumor or a portion thereof is inserted into a hole that substantially matches the tumor's diameters. An additional output pore in the box enables lowering the pressure. The excessive gas is cleared up from the box through this output pore as described above. For example, the gas can flow into the box containing the tumor for 2 seconds followed by a suction of the gas for 2 seconds.

An exemplary delivery system comprises an outer lumen or cannula, trocar, tube, etc. An inner lumen or cannula, tube, etc. can be disposed coaxially inside of the outer lumen. The inner lumen is disposed approximately centrally in the outer lumen, although other configurations are also contemplated.

In some embodiments of such an exemplary delivery system, a space, preferably an exhaust space, is between the outer lumen and inner lumen. The exhaust space can be annular or may take any other configuration and/or geometry. A tip can be attached to the inner lumen at the distal end and is in fluid communication with the inner lumen. In some embodiments, the tip comprises a wire mesh or screen that accesses the space inside of the inner lumen. The delivery device may be advanced to an administration site in the retracted configuration. In some embodiments, the tip is rounded and seals the outer lumen when it is in the retracted position, thus easing insertion of the tip into the body where it is brought adjacent to an administration site.

When the tip of the system is brought adjacent to an administration site, the system is adjusted to its extended configuration in order to affect the administration of the UNO. In the extended configuration, an exhaust path is opened between the distal end of the outer lumen and the tip. UNO is delivered through the inner lumen. The UNO exits the inner lumen at the tip that is in fluid communication with the inner lumen. A wire mesh or screen at the distal end of the tip may assist in diffusing the UNO gas as it exists the device. The exhausted UNO gas returns to the device at the exhaust path. In embodiments, a vacuum is applied to the exhaust space between the outer lumen and the inner lumen in order to attract the exhausted UNO. The exhausted UNO is then brought through the exhaust space to exit the body and be disposed of appropriately. In this way, the device is capable of scavenging UNO from an administration site.

In an alternative configuration, the flow of UNO could be reversed such that UNO is delivered through the space between the outer lumen and inner lumen and removed from the administration site through the inner lumen. In this alternative configuration, a vacuum can be applied to the inner lumen and a positive pressure of UNO is applied to the space defined by the inner and outer lumens. In another alternative, the tip is permanently secured to the outer lumen as well as the inner lumen, such that the tip does not have retracted and extended configurations. In this alternative, permanent passages are provided in the outer lumen for UNO to be expelled from the device or sucked into the device by vacuum. For example, the permanent passages could be small holes or slits radially disposed around the outer lumen, preferably near the distal end of the outer lumen so as to be near the tip of the device.

According to some of any of the embodiments described herein, a delivery device is attached to the end of an endoscope or bronchoscope (e.g., a blue-fluorescence endoscope or bronchoscope) so that the insertion of the delivery device into the body, advancement towards the administration site, and retraction from the body can be visually observed by or otherwise made known to the operator or someone working in concert with the operator. Alternatively, the delivery device can be attached to a guidewire to insert, advance, and retract the device more effectively. Additionally, the device can be coated with a fluoroscopic material or have one or more fluoroscopic tags attached to it so that its insertion, advancement, and retraction could be fluoroscopically observed.

In some embodiments, a system for delivering UNO to and scavenging NOx from, one or more administration sites is intended to fit over the distal end or tip of an endoscope or bronchoscope. The device can be, for example, annular-shaped, having a hole in or about its center and is approximately circular in shape. The hole is preferably sized to accommodate the distal end of an endoscope or bronchoscope. For example, the hole is from about 0.5 cm to about 10 cm in diameter. The hole can be sized so as to fit snugly over the distal end of an endoscope or bronchoscope.

According to some of any of the embodiments described herein, the delivery system comprises a double-needle system in which a suction needle is located adjacent to or proximal to the gas delivery needle (e.g., a distance of between 3 mm and 1 cm). The suction needle can decrease or maintain intra-tumoral pressure. Further, two or more needles can be spaced at least about 2 mm apart. UNO can be delivered at a flow rate of at least about 0.01 LPM as described herein. The needles can be designed to have holes along the length of the needle. In example, the diameter of the holes is about 1 mm and disposed every 2 mm. The needles can be disposed within a lumen, placed outside of the tumor mass, while the shaft of the needle can be placed inside the tissue. The length of the needles can be selected to be at least half the tumor's longest dimension. Vacuuming gas through one or more suction needles or holes can be done in a pulsed or continuous manner, preferably synchronized with UNO administration. For example, the UNO can flow into the tumor for 2 seconds followed by applying a vacuum or suction for 2 seconds. One or more, such as a plurality, of needles or an array of needles, such as nano-sized or micron-sized needles can be used. In some embodiments, the gas is injected into the tumor by means of an array of needles with a spacing of about 0.5 cm. The ratio of suction needles and delivery needles can be 1:10 to 10:1, preferably 1:1. The suction needles can be designed to remove less than about 1 liter per minute per cm gas or fluid.

According to some of the any of the embodiments described herein, UNO is applied to a targeted tumor through one or more intra-tumoral channels. For example, a channel 2 mm in width respective to every 4 mm of tissue can be formed through which gas can be delivered, for example at a flow rate higher than 0.01 LPM, directly to the tumor mass by a needle that is placed at the center of each channel, while the gas is cleared up from a scavenging channel. The flow rate in the scavenging channel can be lower than the delivery channel, such as at least 0.001 LPM less than the UNO delivery flow rate. The vacuuming can be accomplished through suction needles or hoses and can be pulsed or continuous, preferably synchronized with UNO administration. For example, the UNO can flow into the tumor for 2 seconds followed by a suction of the gas for 2 seconds.

According to some of the any of the embodiments described herein, UNO is sprayed onto a targeted tumor, optionally when a method as described herein is used in combination with surgical treatment (e.g., tumor resection) and/or in combination with UNO intra-tumoral injection and/or when the tumor is inoperable, flat or amorphous.

The present invention provides methods to treat cancer by a combined treatment comprising UNO and a checkpoint inhibitor. In some embodiments, the methods further comprise an immune adjuvant.

According to specific embodiments, treatment with UNO and a checkpoint inhibitor has a combined improved anti-cancer or anti-tumor activity. According to specific embodiments, treatment with UNO, a checkpoint inhibitor, and an immune adjuvant has a combined improved anti-cancer or anti-tumor activity.

As used herein the phrase “combined improved anti-cancer or anti-tumor activity” refers to at least additive but also synergistically improved anti-cancer or anti-tumor activity as compared to treatment with each of the agents when administered as a single agent or a combination of two of the agents, which may be determined by the effect on e.g., tumor size, tumor regression, symptoms of the disorder or subject's survival.

The checkpoint inhibitor of some embodiments of the invention can be administered to an organism per se, or in a pharmaceutical composition where it is mixed with suitable carriers or excipients.

The immune adjuvant of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the checkpoint inhibitor and/or the immune adjuvant accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially trans-nasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to specific embodiments, the pharmaceutical composition is administered intravenously.

According to specific embodiments, pharmaceutical composition is administered as an intravenous infusion over 20-60 minutes e.g., 30 minutes after dilution.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternatively, one may administer the pharmaceutical composition in a local rather than systemic manner, for example, via injection of the pharmaceutical composition directly into a tissue region of a patient.

According to specific embodiments, the pharmaceutical composition is administered intra-tumoral or in close vicinity to the tumor.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

In some of any of the embodiments described herein, co-administering of UNO and a checkpoint inhibitor allows the administration of the UNO and/or the checkpoint inhibitor at a sub-therapeutic dosage; which may, for example, reduce the adverse effects of the treatment. In some embodiments, the UNO is administered prior to the checkpoint inhibitor.

In some of any of the embodiments described herein, co-administering of UNO with the checkpoint inhibitor and the immune adjuvant allows the administration of the UNO, the checkpoint inhibitor and/or the immune adjuvant at a sub-therapeutic dosage; which may, for example, reduce the adverse effects of the treatment.

Herein, the term “sub-therapeutic dosage” or “sub-therapeutic dose” refers to a dosage of an agent which is lower than a dosage of the agent effective (when administered alone) to prevent, alleviate or ameliorate symptoms of a disorder (e.g., cancer) or prolong the survival of the subject being treated; for example, a dosage lower than a dosage of the agent recognized in the art to be effective (when administered alone) for such a purpose, or a dosage that was shown to be effective (when administered alone) to prevent, alleviate or ameliorate symptoms of a disorder (e.g., a tumor) or prolong the survival of a specific subject.

In other words, the term “sub-therapeutic dosage” or “sub-therapeutic dose” refers to a dosage of an agent which is lower than a therapeutically effective amount of the agent (when administered alone) to the subject, as defined herein.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to determine useful doses more accurately in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosages for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is affected or diminution of the disease state is achieved.

The amount of a composition to be administered will, of course, be dependent on the subject being treated, the severity of the affliction, the manner of administration, the judgment of the prescribing physician, etc.

According to specific embodiments, the checkpoint inhibitor is administered in a dose of 1-10 mg/kg.

According to specific embodiments, the checkpoint inhibitor is administered in a dose of 100-500 mg (e.g., 200 mg or 400 mg).

Below are non-limiting exemplary known protocols for administration of a checkpoint inhibitor, e.g., pembrolizumab, that can be used with specific embodiments of the invention: Melanoma: 200 mg every 3 weeks or 400 mg every 6 weeks; 2 mg/kg (up to 200 mg) every 3 weeks for pediatrics; NSCLC: 200 mg every 3 weeks or 400 mg every 6 weeks; HNSCC: 200 mg every 3 weeks or 400 mg every 6 weeks; cHL or PMBCL: 200 mg every 3 weeks or 400 mg every 6 weeks for adults; 2 mg/kg (up to 200 mg) every 3 weeks for pediatrics; Urothelial Carcinoma: 200 mg every 3 weeks or 400 mg every 6 weeks; MSI-H or dMMR Cancer: 200 mg every 3 weeks or 400 mg every 6 weeks for adults; 2 mg/kg (up to 200 mg) every 3 weeks for pediatrics; MSI-H or dMMR CRC: 200 mg every 3 weeks or 400 mg every 6 weeks; MSI-H or dMMR Endometrial Carcinoma: 200 mg every 3 weeks or 400 mg every 6 weeks; Gastric Cancer: 200 mg every 3 weeks or 400 mg every 6 weeks; Esophageal Cancer: 200 mg every 3 weeks or 400 mg every 6 weeks; Cervical Cancer: 200 mg every 3 weeks or 400 mg every 6 weeks; HCC: 200 mg every 3 weeks or 400 mg every 6 weeks; MCC: 200 mg every 3 weeks or 400 mg every 6 weeks for adults; 2 mg/kg (up to 200 mg) every 3 weeks for pediatrics; RCC: 200 mg every 3 weeks or 400 mg every 6 weeks as a single agent in the adjuvant setting, or in the advanced setting with either: oaxitinib 5 mg orally twice daily or lenvatinib 20 mg orally once daily; Endometrial Carcinoma: 200 mg every 3 weeks or 400 mg every 6 weeks with lenvatinib 20 mg orally once daily; TMB-H Cancer: 200 mg every 3 weeks or 400 mg every 6 weeks for adults; 2 mg/kg (up to 200 mg) every 3 weeks for pediatrics; cSCC: 200 mg every 3 weeks or 400 mg every 6 weeks; TNBC: 200 mg every 3 weeks or 400 mg every 6 weeks.

In some of any of the embodiments described herein, administration of the UNO may optionally be performed prior to, subsequent to, and/or concomitant with administering the checkpoint inhibitor.

In some of any of the embodiments described herein, administration of the UNO may optionally be performed prior to, subsequent to, and/or concomitant with administering the checkpoint inhibitor and/or the immune adjuvant.

According to specific embodiments, the checkpoint inhibitor is administered prior to the UNO.

According to specific embodiments, the checkpoint inhibitor is administered at least once prior to the UNO followed by administration concomitantly and/or subsequently to the UNO.

According to specific embodiments, the checkpoint inhibitor is administered prior to the immune adjuvant.

According to specific embodiments, the checkpoint inhibitor is administered at least once prior to the immune adjuvant followed by administration concomitant with and/or subsequent to the immune adjuvant.

According to specific embodiments, administration of the UNO is performed prior to administering the checkpoint inhibitor.

According to specific embodiments, the UNO is administered at least 12 hours, at least 16 hours, at least 20 hours, at least 24 hours, at least 30 hours, at least 36 hours, at least 48 hours, at least 56 hours, at least 64 hours, at least 72 hours prior to administration of the checkpoint inhibitor.

According to specific embodiments, the UNO is administered at least 1 day, at least 2 days, at least 3 days, at least 4 days, at least 5 days prior to administration of the checkpoint inhibitor.

According to specific embodiments, the checkpoint inhibitor is administered at least 2, at least 3, at least 4 or at least 5 times.

According to specific embodiments, the checkpoint inhibitor is administered every 1-50 days, every 1-30, every 1-21 days, every 2-10 days or every 2-7 days.

According to specific embodiments, the checkpoint inhibitor is administered every day or every 2 days.

According to specific embodiments, the checkpoint inhibitor is administered every 2 days.

According to specific embodiments, the checkpoint inhibitor is administered every 1-10 weeks, every 2-8 weeks or every 3-6 weeks.

According to specific embodiments, the checkpoint inhibitor is administered every week, every two weeks, every three weeks, every four weeks, every 5 weeks or every 6 weeks.

In some embodiments, UNO is administered alone or in combination with a checkpoint inhibitor in one or more cycles. A cycle is defined as a time period of from 7 to 28 days (e.g., 7 or 14 or 21 or 28 days). In some embodiments, an immune adjuvant is further administered to a subject.

In some embodiments, the cycle is repeated until the subject experiences unmanageable toxicity or disease progression. Subjects can continue in subsequent cycles as long as clinical benefit is observed and demonstrate an objective tumor response.

In some embodiments of any of the cycles, an immune adjuvant is further administered.

According to specific embodiments, the administering comprises:
(i) administering the checkpoint inhibitor; and subsequently
(ii) administering the UNO.

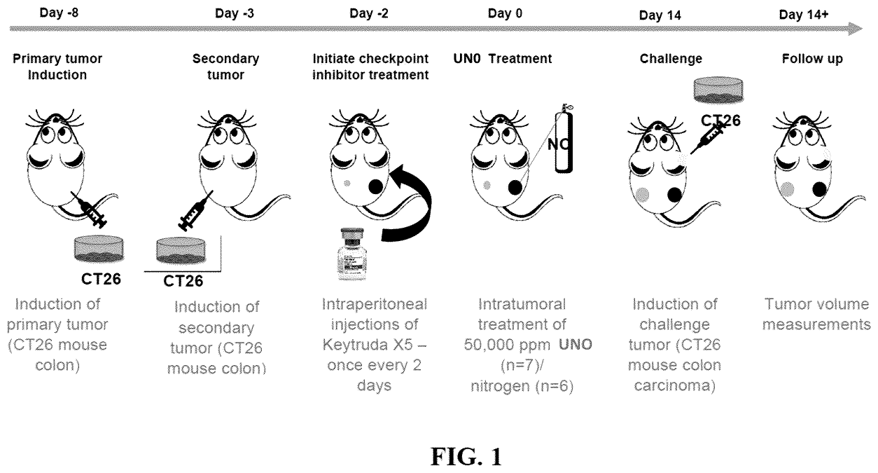

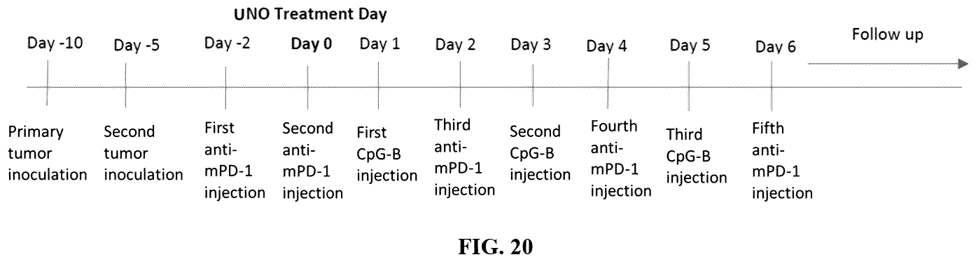

According to specific embodiments, the administering comprises:
(i) administering the UNO; and subsequently
(ii) administering the checkpoint inhibitor.
   Non-limiting exemplary protocol of administration, which can be used with specific embodiments of the invention, are provided in FIG. 1 and FIG. 20.

According to specific embodiments, when an immune adjuvant is used, the checkpoint inhibitor and the immune adjuvant are administered on consecutive days.

According to specific embodiments, the administering comprises:
(i) administering the checkpoint inhibitor; and subsequently
(ii) administering the UNO; and subsequently
(iii) administering the immune adjuvant.

According to specific embodiments, the administering comprises:
(i) administering the checkpoint inhibitor; and subsequently,
(ii) administering the checkpoint inhibitor and the UNO; and subsequently
(iii) administering sequentially the checkpoint inhibitor and the immune adjuvant.

According to specific embodiments, the administering comprises:
(i) administering the UNO; and subsequently
(ii) administering the checkpoint inhibitor; and subsequently
(iii) administering the immune adjuvant.

According to specific embodiments, the administering comprises:
(i) administering the UNO; and subsequently,
(ii) administering the checkpoint inhibitor and the UNO; and subsequently
(iii) administering sequentially the checkpoint inhibitor and the immune adjuvant.

According to specific embodiments, UNO is used as a sensitizing treatment to the checkpoint inhibitor, wherein the UNO is used to upregulate the expression of a target immune checkpoint protein before the administration of the checkpoint inhibitor.

According to some embodiments of the invention, UNO sensitizes the cancer to treatment with a PD-1 inhibitor by upregulating the expression of PD-1 or PD-L1 before the PD-1 inhibitor is administered.

According to some embodiments of the invention, UNO sensitizes the cancer to treatment with a PD-L1 inhibitor by upregulating the expression of PD-L1 before the PD-L1 inhibitor is administered.

According to some embodiments of the invention, UNO sensitizes the cancer to treatment with a CTLA-4 inhibitor by upregulating expression of CTLA-4 before the CTLA-4 inhibitor is administered.

According to some embodiments of the invention, the administration of UNO results in a higher tumor-specific immune cell response. In some embodiments, the higher tumor-specific immune cell response is an increase in tumor antigen-specific CD8+ T-cells.

According to some embodiments of the invention, the combination of UNO and a checkpoint inhibitor results in a higher tumor-specific immune cell response. According to some embodiments of the invention, the combination of UNO and a checkpoint inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a PD-1 inhibitor results in a higher tumor-specific immune cell response. In some embodiments, the higher tumor-specific immune cell response is an increase in tumor antigen-specific CD8+ T-cells. According to some embodiments of the invention, the combination of UNO and a PD-1 inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a PD-L1 inhibitor results in a higher tumor-specific immune cell response. In some embodiments, the higher tumor-specific immune cell response is an increase in tumor antigen-specific CD8+ T-cells. According to some embodiments of the invention, the combination of UNO and a PD-L1 inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a CTLA-4 inhibitor results in a higher tumor-specific immune cell response. In some embodiments, the higher tumor-specific immune cell response is an increase in tumor antigen-specific CD8+ T-cells. According to some embodiments of the invention, the combination of UNO and a CTLA-4 inhibitor synergistically results in a higher tumor-specific immune cell response. In some embodiments, the higher tumor-specific immune cell response is an increase in tumor antigen-specific CD8+ T-cells.

According to some embodiments of the invention, the combination of UNO and a LAG-3 inhibitor results in a higher tumor-specific immune cell response. In some embodiments, the higher tumor-specific immune cell response is an increase in tumor antigen-specific CD8+ T-cells. According to some embodiments of the invention, the combination of UNO and a LAG-3 inhibitor synergistically results in a higher tumor-specific immune cell response. In some embodiments, the higher tumor-specific immune cell response is an increase in tumor antigen-specific CD8+ T-cells.

In some embodiments, UNO can be administered to treat solid tumors at a dose of one of 10,000 ppm, 15,000 ppm, 20,000 ppm, 25,000 ppm, 50,000 ppm, and 100,000 ppm through local administration, such as intra-tumoral injection. In some embodiments, the UNO can be administered for a duration of 5 minutes at a flow rate of 0.2 L/min. In some embodiments, the UNO can be administered for multiple cycles. In some embodiments, a cycle is a three-week period (21 days), wherein the UNO can be administered on day 1 and/or day 8 of one or more 21-day cycles. In some embodiments, a cycle is a four-week period (28 days), wherein the UNO can be administered on day 1 and/or day 8 and/or day 15 of one or more 28-day cycles. In some embodiments, the UNO can be administered in combination with a checkpoint inhibitor, wherein the checkpoint inhibitor is one of a PD-1 inhibitor, PD-L1 inhibitor, a CTLA-4 inhibitor, and a LAG-3 inhibitor. In some embodiments, the checkpoint inhibitor can be administered intravenously subsequent to the administration of the UNO.

In some embodiments, UNO can be administered to treat primary or metastatic tumors at a dose of 50,000 ppm through local administration, such as intra-tumoral injection. In some embodiments, the UNO can be administered for a duration of 5 minutes at a flow rate of 0.2 L/min. In some embodiments, the UNO can be administered for multiple 21-day cycles, wherein the UNO can be administered on day 1 of one or more 21-day cycles.

In some embodiments, UNO can be administered to treat triple negative breast cancer (TNBC) at a dose of 50,000 ppm through local administration, such as intra-tumoral injection. In some embodiments, the UNO can be administered for a duration of 5 minutes at a flow rate of 0.2 L/min. In some embodiments, the UNO can be administered for multiple 21-day cycles, wherein the UNO can be administered on day 1 of one or more 21-day cycles. In some embodiments, the UNO can be administered in combination with a checkpoint inhibitor, wherein the checkpoint inhibitor is a PD-1 inhibitor. In some embodiments, the PD-1 inhibitor is pembrolizumab. In some embodiments, the pembrolizumab can be administered intravenously subsequent to the administration of the UNO. In some embodiments, the pembrolizumab is administered at a dose of 200 mg for a duration of 30 minutes, every three weeks. In some embodiments, the pembrolizumab is administered at a dose of 400 mg for a duration of 30 minutes, every six weeks.

In some embodiments, UNO can be administered to treat advanced cutaneous malignant melanoma at a dose of 25,000 ppm or 50,000 ppm through local administration, such as intra-tumoral injection. In some embodiments, the UNO can be administered for a duration of 5 minutes at a flow rate of 0.2 L/min. In some embodiments, the UNO can be administered for multiple 21-day cycles, wherein the UNO can be administered on day 1 of one or more 21-day cycles. In some embodiments, the UNO can be administered in combination with a checkpoint inhibitor, wherein the checkpoint inhibitor is a CTLA-4 inhibitor. In some embodiments, the CTLA-4 inhibitor is ipilimumab. In some embodiments, the ipilimumab is administered intravenously subsequent to the administration of the UNO, wherein the ipilimumab is administered at a dose of 3 mg/kg for a duration of 30 minutes. In some embodiments, the ipilimumab is administered for up to four cycles.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term throughout “about” as used herein when referring to a measurable value such as an amount of weight, time, dose, etc. is meant to encompass variations of +/−20% or +/−10% from the specified amount, as such variations are appropriate to perform the disclosed method. In embodiments, the term “about” is meant to encompass variations of +/−5%. In embodiments, the term “about” is meant to encompass variations of +/−1%. In embodiments, the term “about” is meant to encompass variations of +/−0.1%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion. Generally, the nomenclature used herein, and the laboratory procedures utilized in the present invention include molecular, biochemical, microbiological and recombinant DNA techniques.

### Colon Cancer Model

### Materials and Methods

Preparation of cancer cells for injection—Mouse colon carcinoma CT26.WT cells (ATCC, Cat no. CRL-2638) suspended in Hanks' Balanced Salt Solution (HBSS) (Biological Industries, Israel), at a concentration of 5.0×10⁶ cells/ml were freshly prepared on the day of cancer model induction. Cells were grown to 70% confluency and were harvested using trypsin (Biological Industries, Israel), and counted using a cell counter device. The cells were then centrifuged at 1,200 rpm (241×g) for 8 minutes, and the pellet was re-suspended in ice cold HBSS at 5.0×10⁶ in 1 ml (5.0×10⁵ cells in 100 μl solution). Cell viability was >90% as determined by trypan blue staining prior to inoculation to mice. At the point of inoculation, cells were at passage 10 at most.

Inoculation of CT26 cancer cells to mice—100 μl/mouse of the CT26 cancer cells suspension (concentration of 5.0×10⁶ cells/ml) were inoculated subcutaneously (s.c.) to the right flank of male BALB/c mice, 9 weeks of age (Envigo, Israel). Five days later, CT26 cells at the same dose were inoculated s.c., to the left flank of each mouse. Eight days post primary tumor inoculation tumor-bearing mice were divided into the following groups (day −2) as depicted in Table 1 below and treated according to the timeline shown in FIG. 1.

TABLE 1

| Treatment | No. of mice |
|---|---|
| N₂ + pembrolizumab (Keytruda) | 6 |
| UNO + pembrolizumab (Keytruda) | 7 |

On day 14, CT26 cancer cells suspension (concentration of 5.0×10⁶ cells/ml) were inoculated underneath the upper right arm of all animals (challenge tumor) at a dose volume of 0.1 ml (total 500,000 cells per mouse).

Mice were observed for a total of 100 days. During the experiment, mice were assessed for eight clinical signs, and the primary and secondary tumors were measured. Viability tests, for mortality and morbidity, were performed 2-3 times a week on regular working days. Clinical sign observations included changes in skin, fur, eyes, mucous membranes, occurrence of secretions and excretions. Presence of bizarre behavior was also observed and recorded. Complete regression was defined as mice whose primary tumor was fully regressed (disappeared); partial regression was defined as mice whose primary tumor volume was smaller by at least 80% from the average tumor volume of the non-treated group.

Mice were sacrificed and excluded from the study if they showed severe pain and enduring signs of severe distress or if a decrease of body weight was greater than 20% from initial body weight, or if their total tumor volume was >1500 mm³. When indicated, data was compared to untreated mice, as taken from previous experiments using the same tumor model (also referred to herein as historical data); and to treatment with pembrolizumab alone, as taken from the literature (see https://pubmed(dot)ncbi(dot)nlm(dot)nih(dot)gov/27799536/).

UNO treatment—On the day of treatment (day 0), mice were anesthetized by an intraperitoneal (i.p.) injection of 100 mg/kg ketamine and 10 mg/kg xylazine hydrochloride solution. Tumor-bearing mice were treated with 50,000 ppm of UNO delivered into the tumor by a 23 G needle over 5 minutes, at 0.2 LPM. After treatment, all mice were warmed and closely monitored until complete recovery was observed.

N₂ treatment—On the day of treatment (day 0), mice were anesthetized by an intraperitoneal (i.p.) injection of 100 mg/kg of ketamine and 10 mg/kg of xylazine hydrochloride solution. Tumor-bearing mice were treated with N₂ delivered into the tumor by a 23 G needle over 5 minutes, at 0.2 LPM. After treatment, all mice were warmed and closely monitored until complete recovery was observed.

Pembrolizumab treatment—Pembrolizumab (Keytruda, Trilog Cat No. 7006873300) was administered 4-6 times intraperitoneally (i.p.) at a dose of 10 mg/kg, every other day from day −2. The injections were administered to approximately the same location each time.

### Example 1

### The Effect of Combined Treatment of UNO and Pembrolizumab on Primary and Secondary Tumors

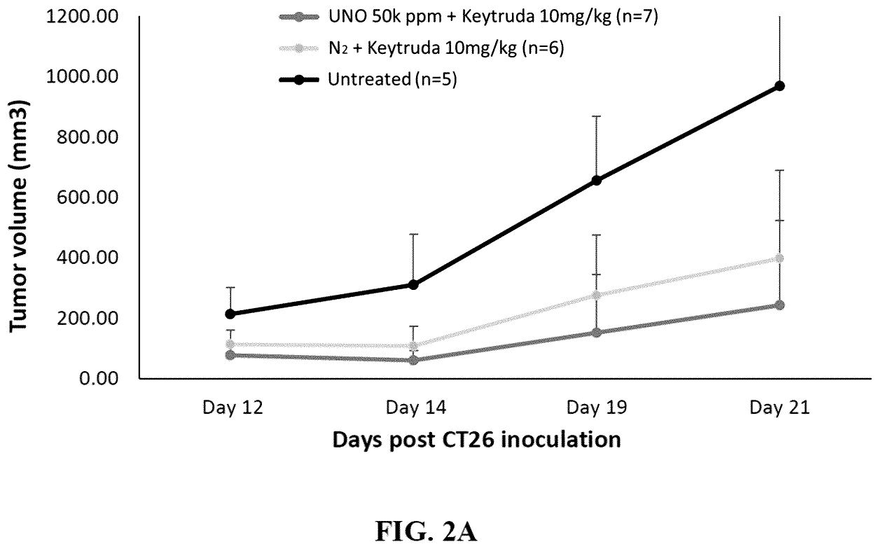

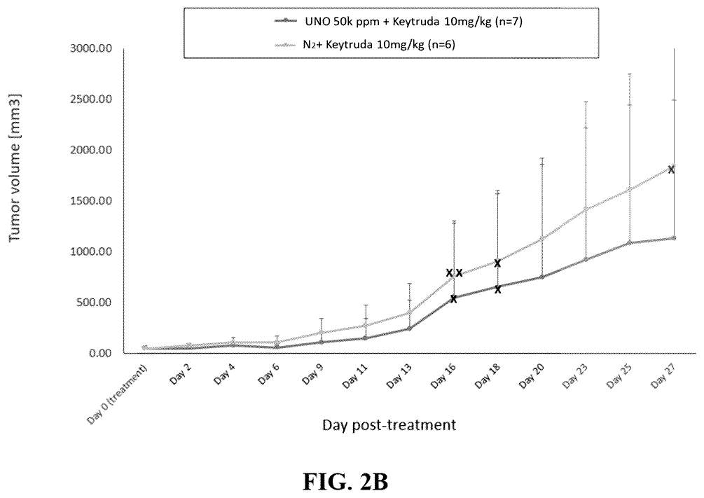

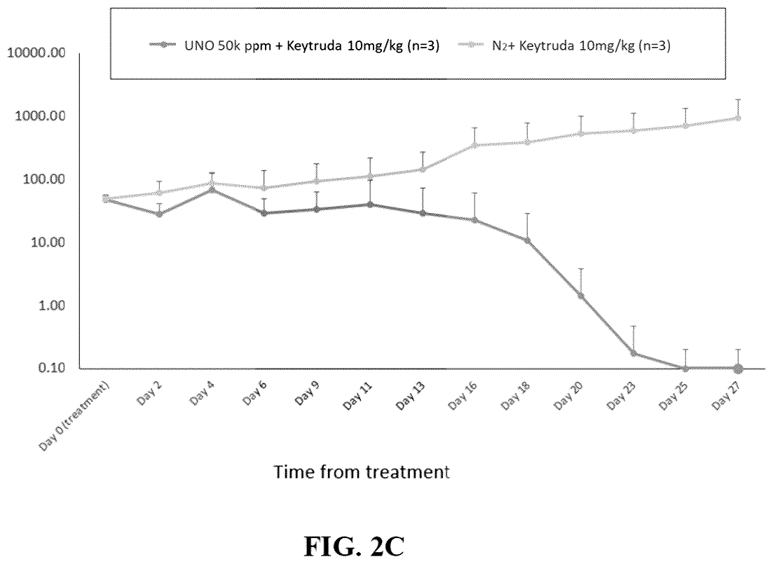

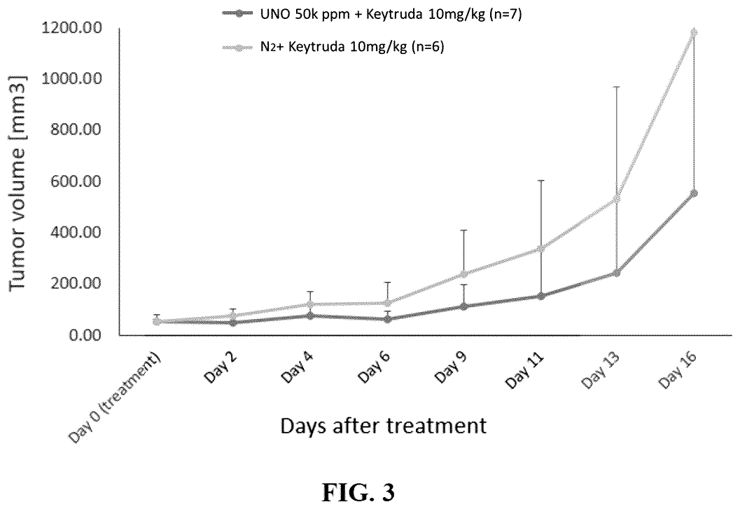

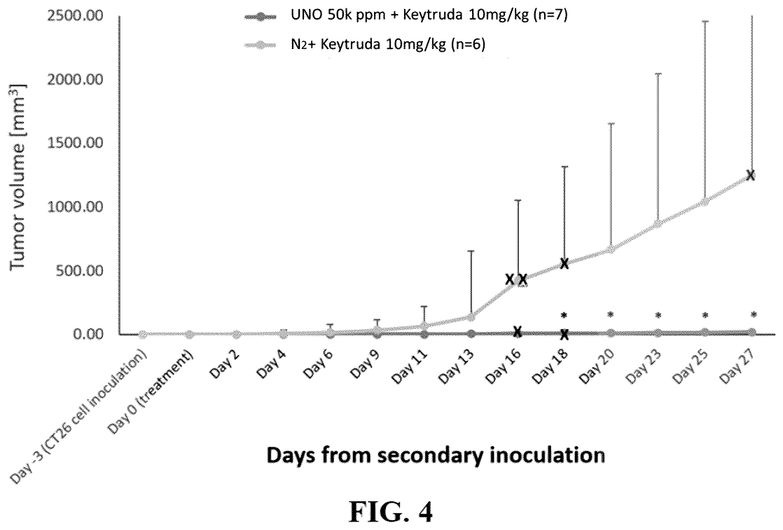

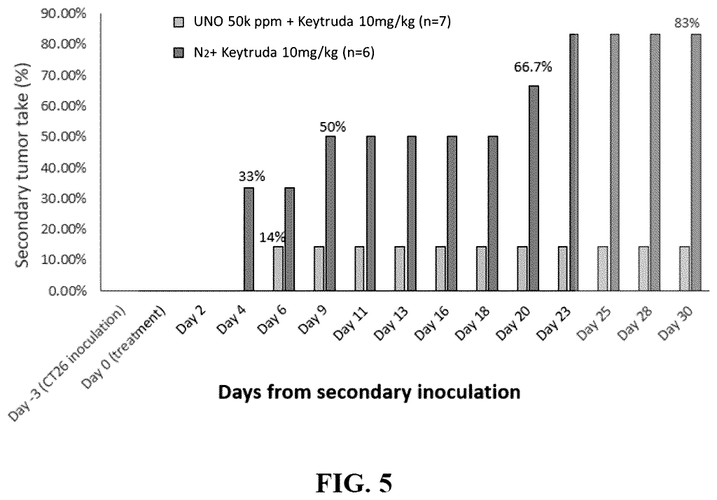

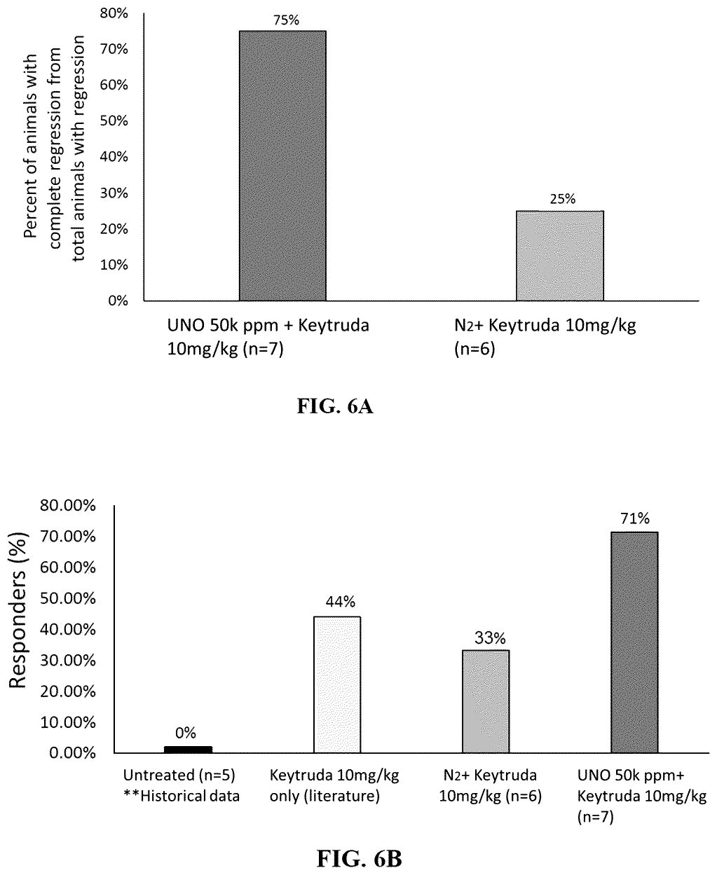

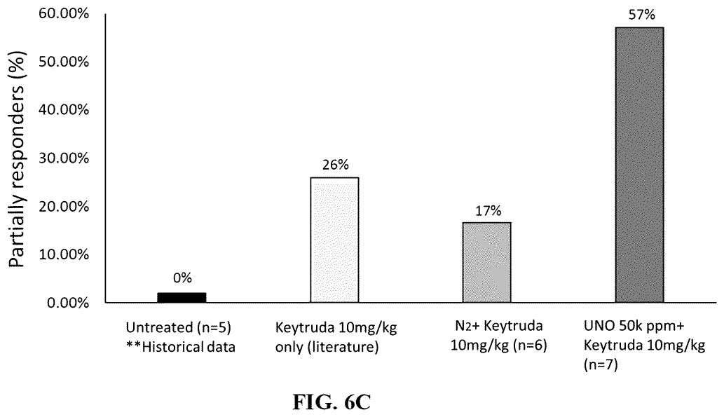

Primary and secondary tumors of mice treated with UNO in combination with pembrolizumab (see FIG. 1) were significantly smaller than tumors of untreated mice or mice treated with N₂ in combination with pembrolizumab (FIGS. 2C and 3-4). Further, the combined treatment of UNO and pembrolizumab increased the rejection rate of the secondary tumor as compared to treatment with N₂ in combination pembrolizumab (FIG. 5). In addition, the combined treatment of UNO and pembrolizumab showed the highest percentage of primary and secondary tumors complete and partial regression (FIGS. 6A-C).

### Example 2

### The Effect of Combined Treatment with UNO and Pembrolizumab on Mice Survival

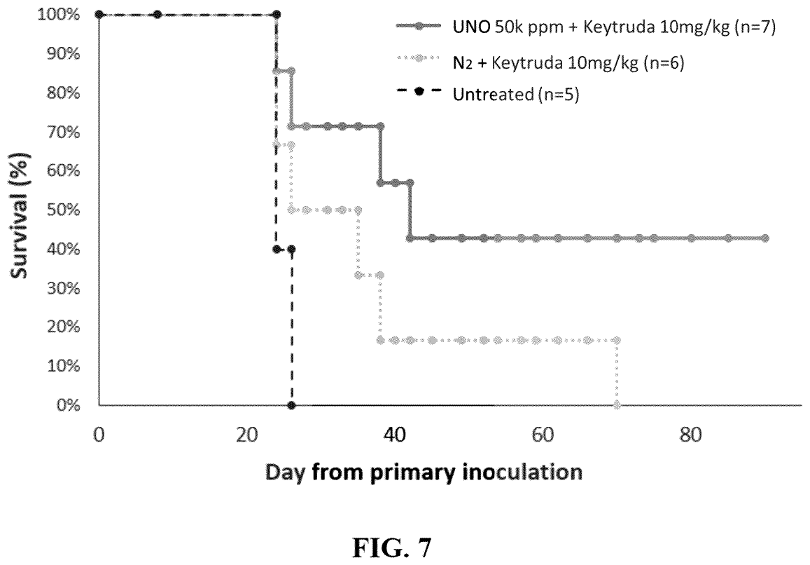

As shown in FIG. 7, the combined treatment of UNO and pembrolizumab had a pronounced effect on mice survival compared to untreated mice or mice treated with N₂ and pembrolizumab. Mice treated with UNO+pembrolizumab survived for 100 days after primary tumor induction (40%) as opposed to the N₂+pembrolizumab group in which all mice died by day 70 after primary tumor induction.

### Example 3

NO gas: 25,000 ppm-100,000 ppm of UNO was administered from 2.9 L cylinders, with N₂ serving as its stabilizing gas (Gordon Gas and Chemical, Tel Aviv, Israel). All procedures were performed in a chemical hood. Gases were delivered via a pressure regulator through a PVC hose (International Biomedical, USA). The flow rate was set to 0.2 (for in vivo studies)-1.0 (for in vitro studies) liters per minute (LPM) using a manual flow meter.

Tumor cell lines: Mouse CT26.WT colon cell line was purchased from the American Type Culture Collection (ATCC) local distributor, Sartorius (Beit Haemek, Israel). CT26 cell line was grown in RPMI-based media (ATCC) supplemented with 10% fetal bovine serum and 1% penicillin-streptomycin (Sartorius).

Preparation of tumor cells: Tumor cell suspensions were prepared in a cell culture medium or Hanks' Balanced Salt Solution (HBSS, Sartorius) at concentrations of 1×10⁵ cells/ml for in vitro studies or 5.0×10⁶ cells/ml for in vivo studies. Freshly prepared cells were grown to 70% confluency, harvested using trypsin (Sartorius), and counted using a hemocytometer.

In vivo experiments: CT26 cells were inoculated subcutaneously (s.c.) on the right flank of 8-10-week-old female and male Balb/c mice (Envigo, Israel) at a concentration of 5.0×10⁵ CT26 cells in 100 μL HBSS. Treatments were initiated after tumors reached an average volume of ˜80 mm³ (usually 8 days following tumor inoculation). Mice were evaluated for tumor volume using a digital caliper.

In vivo UNO treatment: Before each treatment, mice were anesthetized by an intraperitoneal (i.p.) injection of 100 mg/kg of ketamine (Zoetis) and 10-20 mg/kg of xylazine hydrochloride solution (Abic). After 10 minutes, mice were treated with intra-tumoral delivery of 50,000 ppm of UNO. The needle was inserted into the tumor horizontally and located approximately in the center (about half of the tumor diameter, depending on tumor size and shape). Gas was injected for 5 or 10 minutes using a 23 G needle. The outlet pressure was set to ˜2 bar using a pressure regulator connected to the cylinder. A stainless-steel PTFE-coated hose was connected to the pressure regulator and a manual flow meter on the other end. A PVC hose was connected to the flow controller and a 23 G hypodermic needle. The treatment regimen was 50,000 ppm of UNO at a 0.2 liter/minute rate for 5 or 10 minutes using a manual flow meter.

Anti-PD-1 treatment regimen: Up to five doses of 5-10 mg/kg of a murine anti-PD-1 or anti-mPD-1 (BioXcell, RMP1-14 BP0146, LOT-810421N1) was injected i.p. every two days starting from 2 days prior to treatment with UNO.

Anti-CTLA-4 treatment regimen: Up to five doses of 5 mg/kg of a murine anti-CTLA-4 or anti-mCTLA-4 (BioXcell, BE0131-100) was injected every three days starting 0 or 1 day after UNO treatment. each single mouse, weighing approximately 20 g, will be administrated with ˜0.1 mg anti-mCTLA-4 in a volume of 0.2 ml using a 1 ml syringe with a 27 G needle (0.10 mg×5 injections=0.5 mg total).

Tumor volume calculation: Local tumor growth was determined by measuring 3 mutually orthogonal tumor dimensions 2-3 times per week, according to the following formula:

Tumor Volume=π/6×[Diameter1×Diameter2×Diameter3]

Challenge tumor inoculation: Two days prior to UNO treatment of the primary tumor, the appropriate cancer cell suspensions were prepared, and s.c. cell inoculation was repeated on the contralateral (left) flank. The appearance of a second induced tumor (challenge tumor) was monitored 2-3 times a week by visual and palpable observation.

Statistical analysis: Statistical analysis was performed using Excel (Microsoft, USA) or GraphPad Prism 9.3.1 (GraphPad Software, USA) with P<0.05 considered statistically significant unless stated otherwise.

Combining UNO with anti-PD-1 Slows CT26 Primary Tumor Growth In Vivo

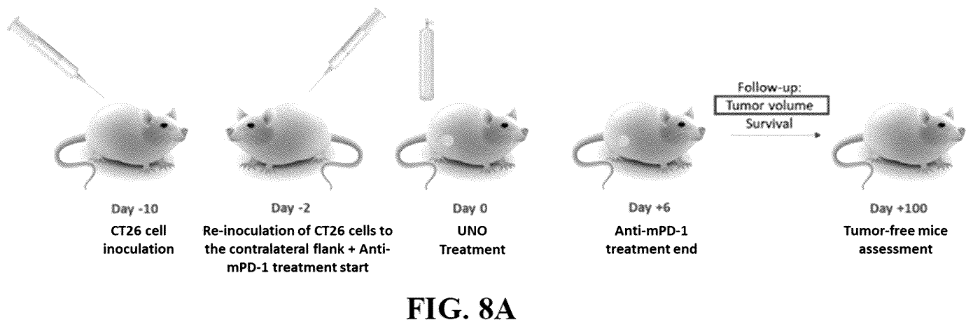

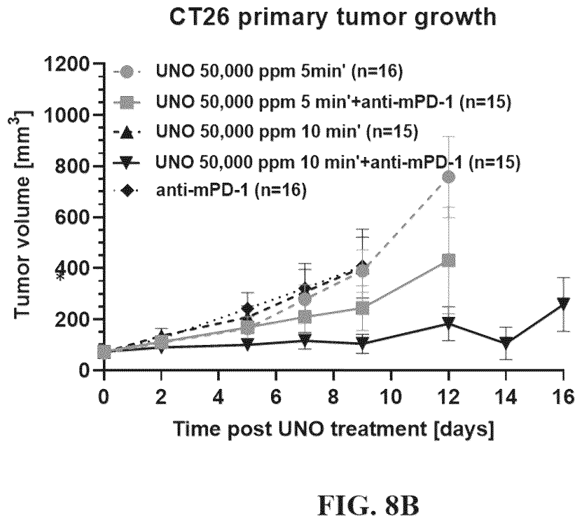

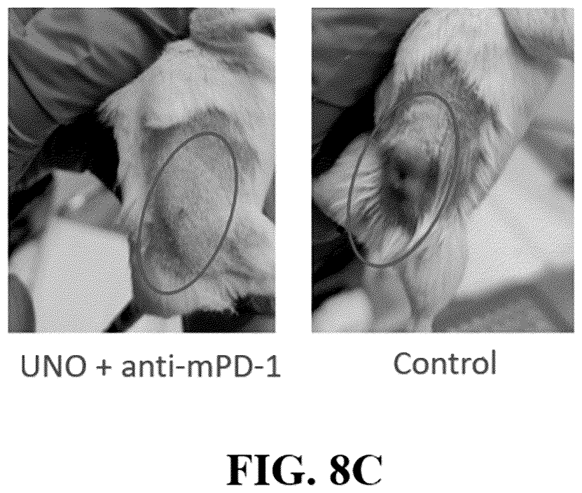

In vitro findings showing that high concentration UNO leads to the upregulation of PD-L1 on CT26 cells (see below) suggest that these cells would now be more susceptible to the effects of anti-PD-L1 or anti-PD-1 checkpoint inhibitors. To test this hypothesis, in vivo testing combining UNO with anti-PD-1 antibody treatments was performed. CT26 cells were injected into the flank of immunocompetent mice. When tumors reached an average size of 50-100 mm³, tumors were intratumorally injected with 50,000 ppm of UNO (n=15-16 for each group) for 5 or 10 minutes, and tumor size was monitored (FIG. 8A). Anti-mPD-1 dosing started two days before UNO treatment. As controls, mice were treated with each therapy alone. Mice treated with 5 or 10 minutes of UNO gas exposure in combination with anti-mPD-1 presented slower tumor growth than those treated with each therapy alone. In addition, the average tumor volume of mice treated with 50,000 ppm of UNO for 10 minutes+anti-mPD-1 was significantly smaller than that of animals treated with anti-mPD-1 alone 9 days post-treatment (FIGS. 8B and 8C, p=0.0005).

These results indicate that combining short-term intra-tumoral UNO administration with the systemic administration of anti-PD-1 results in significant primary tumor growth inhibition compared to each agent alone.

Primary Tumor Regression and Reduced Susceptibility to Secondary Tumors Following UNO Intra-Tumoral Treatment Combined with Systemic anti-PD-1 Administration

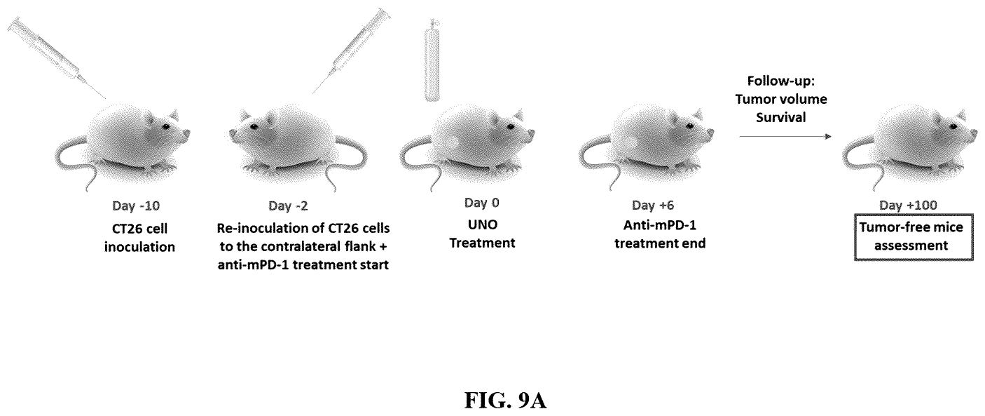

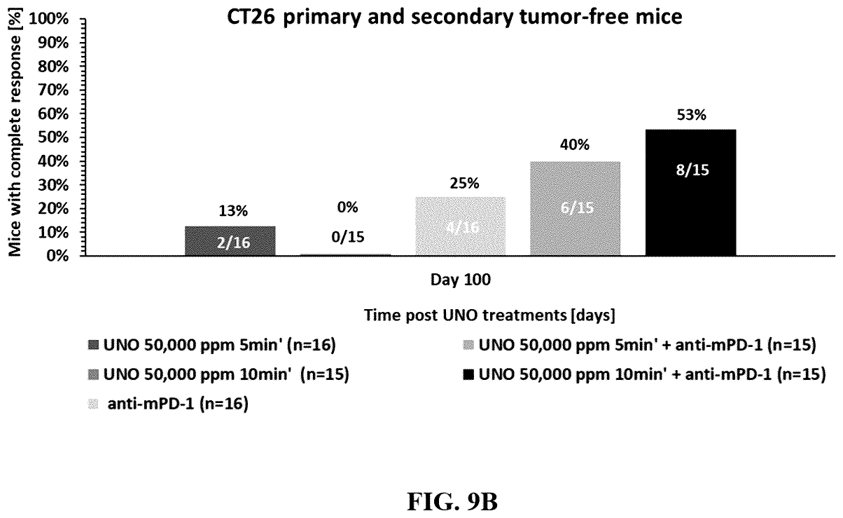

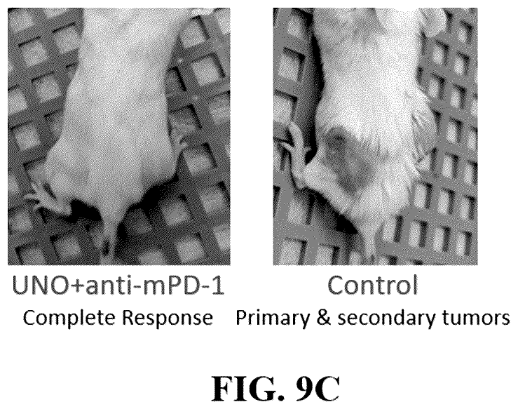

In addition to the significant short-term local effect of UNO treatment on the primary tumor, UNO treatment reduced the growth of both primary and secondary tumors for up to 100 days. Two days before the UNO treatment (at 50,000 ppm) of the primary tumor, a second CT26 cell inoculation was applied on the contralateral flank, and anti-mPD-1 treatment was initiated (FIG. 9A). Importantly, the secondary tumor was induced before the gas treatment, thereby mimicking distant metastatic development and enabling testing of the UNO and anti-mPD-1 combination for a potential abscopal effect.

Primary tumor regression was observed in 53% of the mice treated with UNO and anti-mPD-1. Furthermore, these mice were also free of secondary tumors, an effect that was maintained for up to 100 days after the UNO treatment (FIG. 9B).

Mice Survival is Substantially Prolonged when Treated with 10 Minutes of UNO and anti-PD-1 Up to 100 Days after UNO Treatment

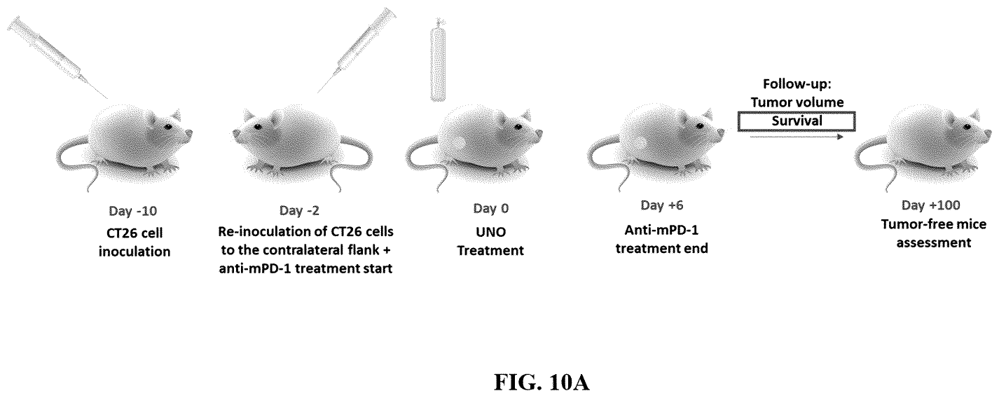

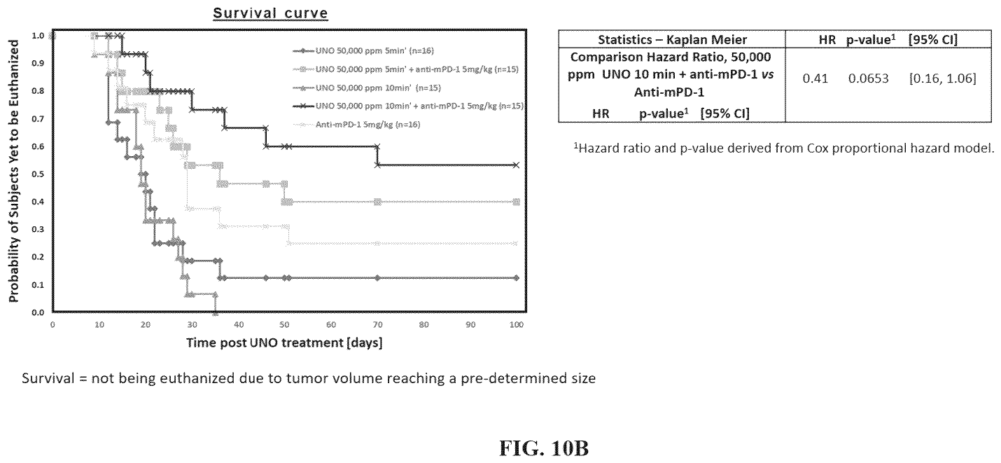

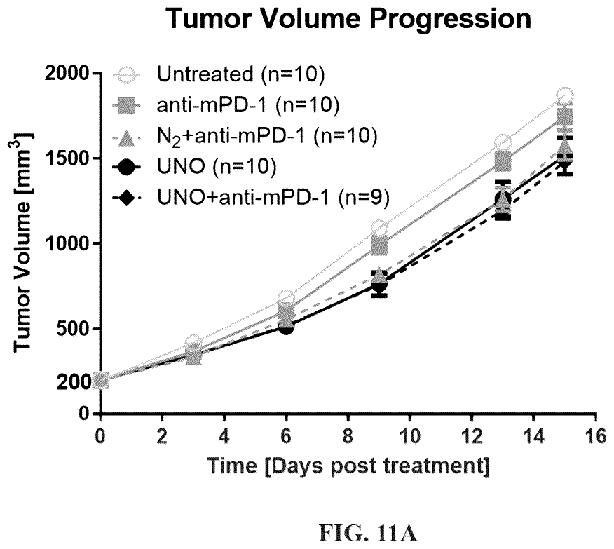

Mice survival was monitored for 100 days after the UNO treatment (see FIG. 10A). Life expectancy was considerably prolonged in mice treated with UNO+anti-PD-1 compared to those treated with anti-PD-1 (P=0.065, FIG. 10B).

### Breast Cancer Model

For the majority of metastatic triple-negative breast cancer (mTNBC) patients, chemotherapy is the standard first-line treatment, however, outcomes in this population are poor. Anti-PD-1 treatment has certain antitumor activity in patients with mTNBC. The ability of UNO to improve the efficacy of anti-PD-1/anti-CTLA-4 antibodies treatment in the aggressive mouse breast cancer model 4T1 was investigated.

### Materials and Methods

Preparation of 4T1 cells: Cancer cell suspension, in Hanks' Balanced Salt Solution (HBSS) (Biological Industries, Israel), at a concentration of 5.0×10\hat{ }6 cells/ml was freshly prepared on the day of the cancer model induction. Cells were grown to a 70-80% confluency and harvested using trypsin. The cells were centrifuged at 1,200 revolutions per minute (rpm)—241 G for 8 minutes, then the pellet was re-suspended in 1 ml of HBSS. The cells were counted using an automated cell counter and a suspension with a concentration of 5.0×10⁶ cells/ml was prepared. Cell viability must be above 90% before cell inoculation to the mice, otherwise new cells will need to be prepared. At the point of inoculation, cells will be at passage 8 at most. The cell suspension was then aspirated into a 1 ml syringe with a 27 G needle, for subcutaneous (s.c.) injection. All injection volume was used and was not re-introduced into the stock solution.

Inoculation of 4T1 cancer cells to mice—Cancer cell suspension at a concentration of 5.0×10⁶ cells/ml was inoculated to the right flank of animals at a volume of 100 μl per mouse. Female Balb/c mice were inoculated with 100 μl of 5.0×10⁶ 4T1 cells/ml. Administration was performed as soon as possible following cell preparation and after manual shaking prior to withdrawal of cell suspension. Injections were performed using a 1 ml syringe and a 27 G needle where withdrawal is done without the needle.

UNO and N₂ treatment—On the day of treatment, mice were anesthetized by an intraperitoneal (i.p.) injection of 100 mg/kg of ketamine and 10 mg/kg of xylazine hydrochloride solution. Tumor-bearing mice were treated with either N₂ or UNO at 50,000 ppm or 100,000 ppm. UNO at 50,000 ppm was administered intratumorally by a 23 G needle for 10 minutes, at 0.2 LPM and 2 bar exit pressure. UNO at 100,000 ppm was administered intratumorally by a 23 G needle for 2 minutes, at 0.2 LPM and 2 bar exit pressure. After treatment, all mice were warmed and closely monitored until complete recovery was observed.

Injection of anti-PD-1—Anti-PD-1 was administered 5 times intraperitoneally (i.p.) at a dose of 10 mg/kg, i.p., using a 1 ml syringe with a 27 G needle. The injection was administered to approximately the same location each time.

Injection of anti-CTLA-4—Anti-CTLA-4 was administered up to 5 times intraperitoneally (i.p.) at a dose of 5 mg/kg, i.p., using a 1 ml syringe with a 27 G needle. The injection was administered to approximately the same location each time.

UNO/Anti-PD-1 Single Dose Study—Balb/c mice (48) were inoculated with mouse breast cancer carcinoma (4T1) cells injected subcutaneously (s.c.) to the right flank. When the average tumor volume reached 200-250 mm³, mice were randomized in groups and were treated with 5% UNO (50,000 ppm), stabilized in N₂, injected intratumorally (i.t.) for 10 minutes at a flow rate of 0.2 liters per minute (LPM) or with 1 ppm of N₂ injected intratumorally (i.t.) for 10 minutes at a flow rate of 0.2 LPM as a control. The mice were administered the gas treatment, e.g., UNO or N₂, once. Mice assigned to the combination treatment group (i.e., gas+anti-mPD-1) were administered anti-mPD-1 injections starting from the day of 1^{st} gas treatment and administered every three days with a final dose of anti-mPD-1 injected at day 12 after the first gas treatment.

Anti-mPD-1 dosing started one day after UNO treatment. The antibody was administered every three days for a total of five injections. The primary tumor volume was assessed using a digital caliper using a standard formula. In addition, mice survival was evaluated. Mice were routinely weighed and monitored for health conditions using mouse distress scoring. Mice reaching a tumor volume of ≥1500 mm3 and/or a score of 12 were humanely euthanized, and the time of death was recorded.

UNO/Anti-PD-1 Repeated Dose Study—Balb/c mice (96) were inoculated with mouse breast cancer carcinoma (4T1) cells injected subcutaneously (s.c.) to the right flank. When the average tumor volume reached 50-150 mm³, mice assigned to gas treatment groups were treated with 5% UNO (50,000 ppm), stabilized in N₂, injected intratumorally (i.t.) for 10 minutes at a flow rate of 0.2 liters per minute (LPM) or with N₂ injected intratumorally (i.t.) for 10 minutes at a flow rate of 0.2 LPM as a control. The mice were administered the gas treatment, e.g., UNO or N₂, twice, on day 9 post-tumor cells inoculation and, 5 days later, on day 14 post-tumor inoculation. Mice assigned to the combination treatment group (i.e., gas+anti-mPD-1) were administered anti-mPD-1 injections starting from the day of first gas treatment and administered every three days thereafter, with a final dose of anti-mPD-1 injected 12 days after the first gas treatment.

Primary tumor volume progression was monitored twice a week by measuring the tumor using a digital caliper calculated as:

$$Tumor volume = \frac{π}{6} \times [ Diameter 1 \times Diameter 2 \times Diameter 3 ] ;$$

where Diameter 1 is length, Diameter 2 is width, and Diameter 3 is height.

Mice receiving two UNO treatments were divided into two groups: in the first group, the primary tumors were surgically removed 15 days after the first UNO treatment, while, in the second group, the tumors were not resected. Mice survival was evaluated. Mice that reached a tumor volume of ≥1500 mm³ and/or a score of 12 were humanely euthanized, and the time of death was recorded. Metastases development was evaluated postmortem.

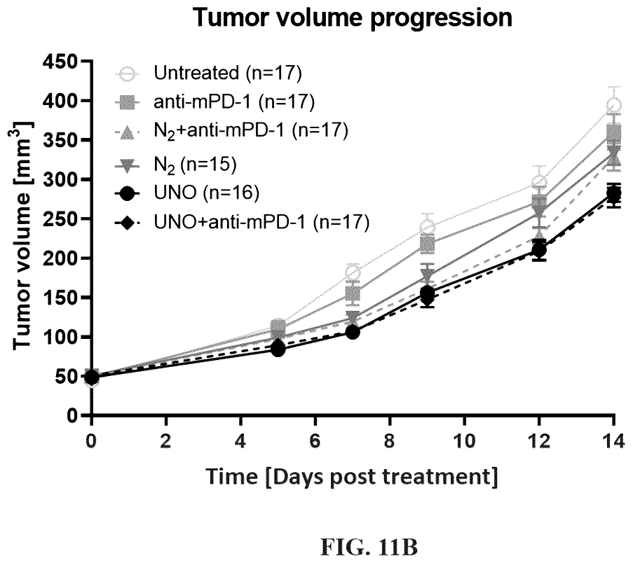

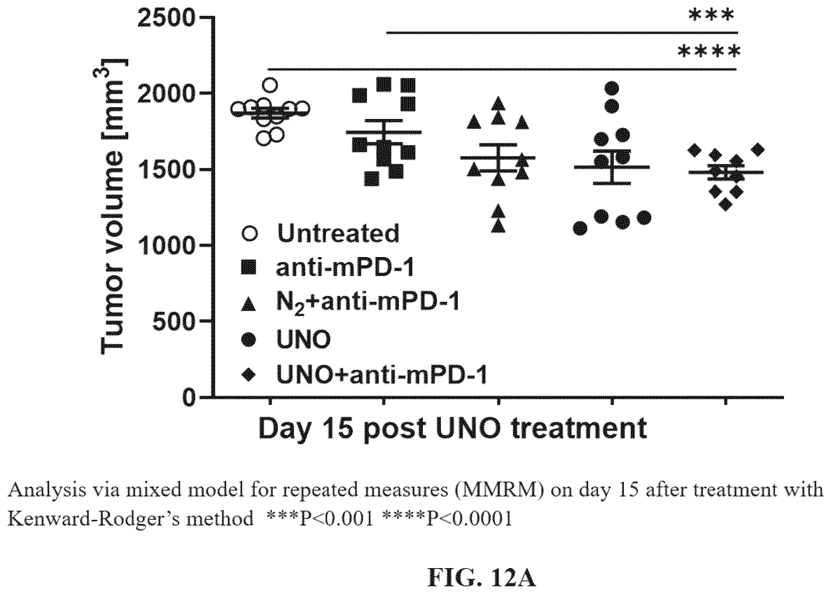

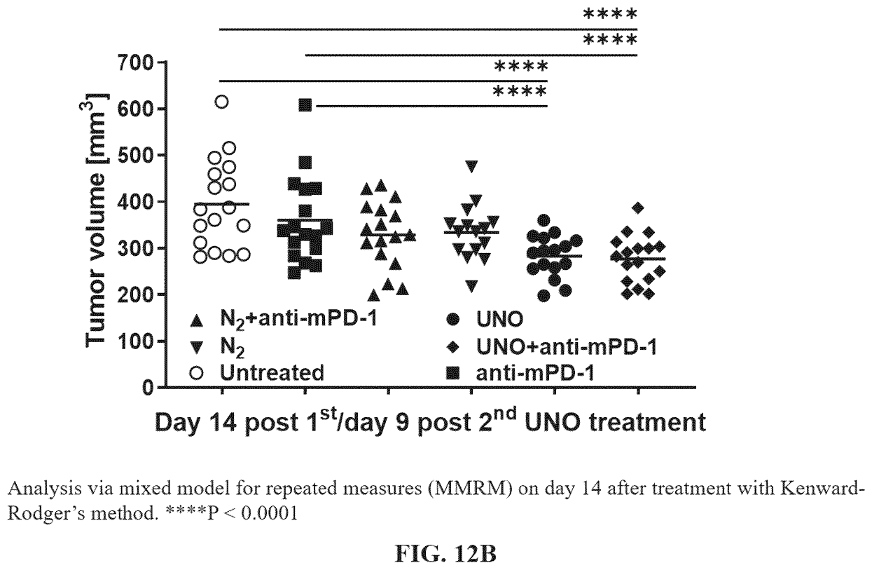

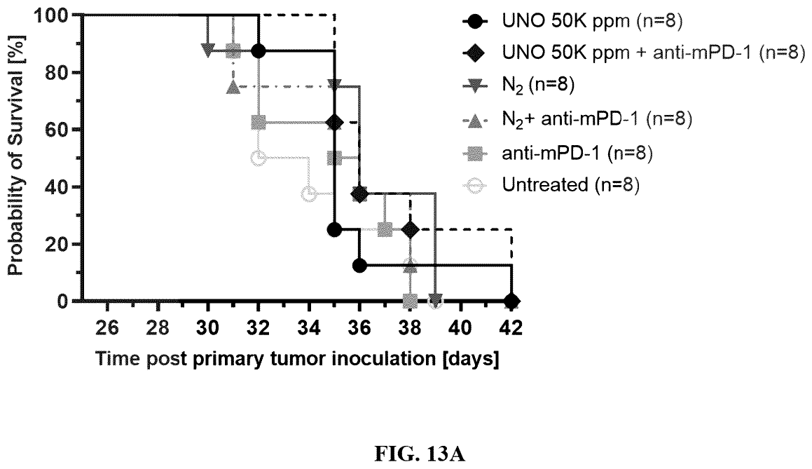

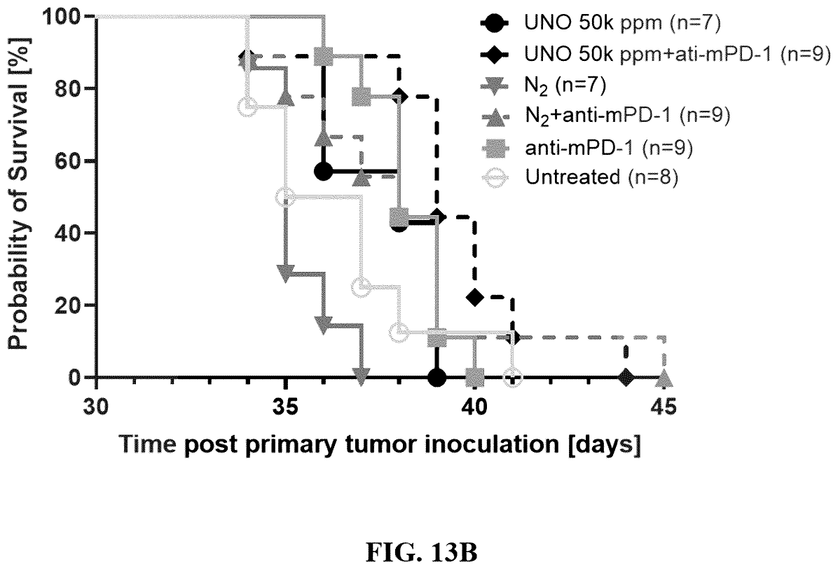

Results—Treating 4T1 tumors with single and repeated doses of UNO improved outcomes compared to anti-PD-1 alone (see FIGS. 11A-13B). Average tumor volume was significantly smaller on day 14 after the first UNO treatment, and Kaplan-Meier curves revealed a tendency toward prolonged survival. These results suggest that local short-term treatment with UNO can serve as a treatment option for cancer patients with tumors that are not amenable to checkpoint inhibitor treatment.

UNO/Anti-CTLA-4 Repeated dose study—Balb/c mice (60) were inoculated with mouse breast cancer carcinoma (4T1) cells injected subcutaneously (s.c.) to the right flank. When the average tumor volume reached 150-200 mm³, mice assigned to gas treatment groups were treated with 10% UNO (100,000 ppm), stabilized in N₂, injected intratumorally (i.t.) for 2 minutes at a flow rate of 0.2 liters per minute (LPM) or with N₂ injected intratumorally (i.t.) for 2 minutes at a flow rate of 0.2 LPM as a control. The mice were administered the gas treatment, e.g., UNO or N₂, twice, on day 11 post-tumor cells inoculation and, 3 days later, on day 14 post-tumor inoculation. Mice assigned to the combination treatment group (i.e., gas+anti-mCTLA-4) were administered anti-mCTLA-4 injections starting one day after the first gas treatment and administered every three days thereafter, with a final dose of anti-mCTLA-4 injected 13 days after the first gas treatment.

Primary tumor volume progression was monitored twice a week by measuring the tumor using a digital caliper calculated as:

$$Tumor volume = \frac{π}{6} \times [ Diameter 1 \times Diameter {2}^{2} ] ;$$

where Diameter 1 is length, Diameter 2 is width.

Mice survival was evaluated. Mice that reached a tumor volume of ≥1500 mm³ and/or a score of 12 were humanely euthanized, and the time of death was recorded. Metastases development was evaluated postmortem.

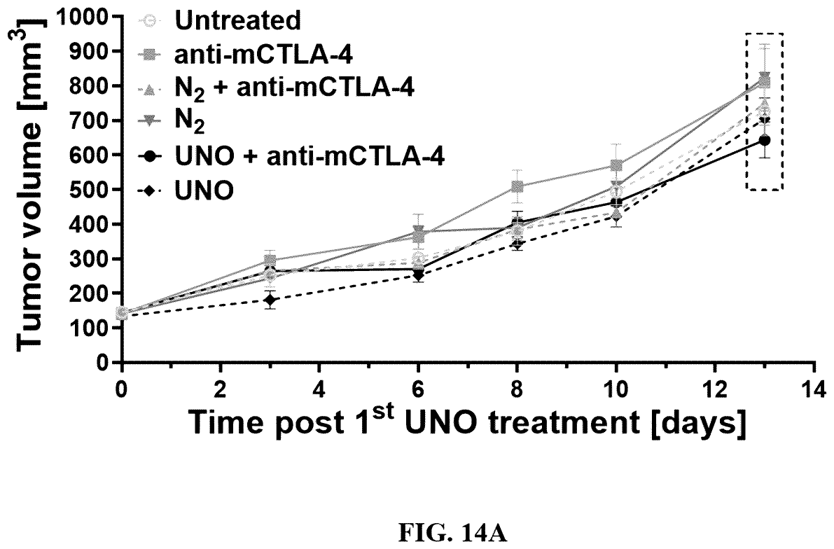

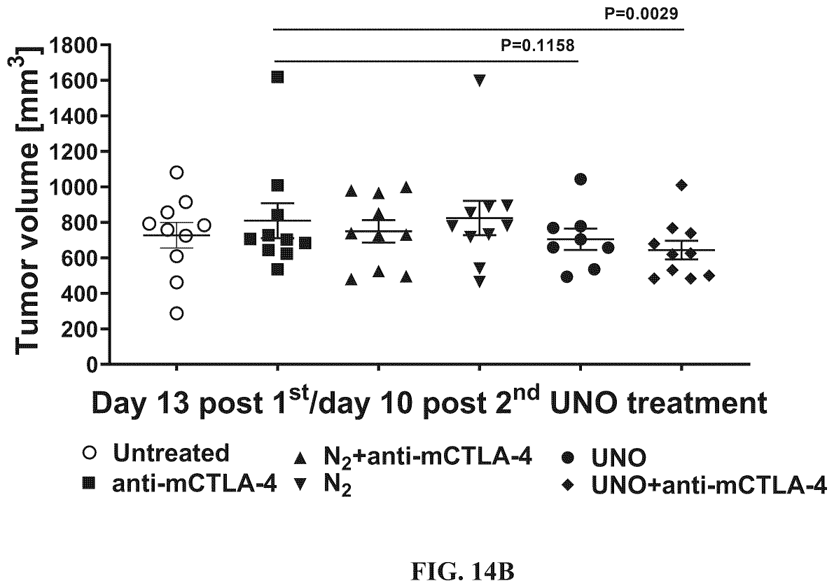

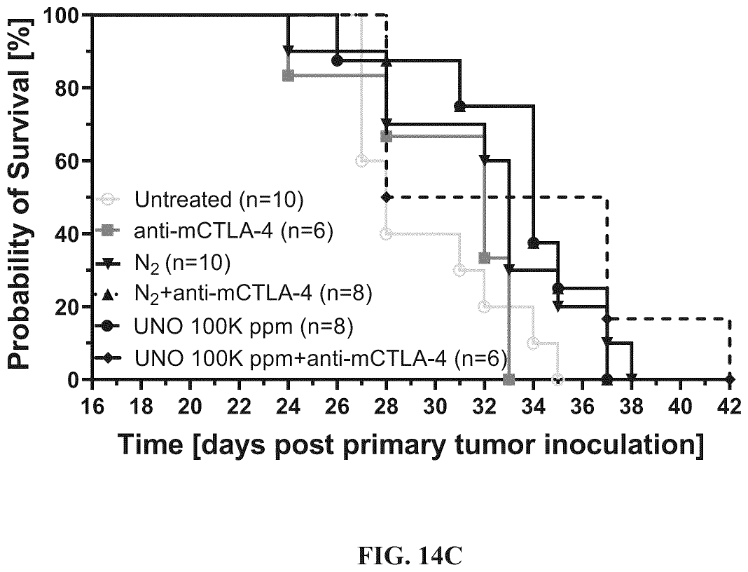

Results—Treating 4T1 tumors with repeated doses of UNO improved outcomes compared to anti-CTLA-4 alone (see FIG. 14). Average tumor volume of mice treated with UNO with or without treatment was smaller on day 13 after the first UNO treatment (10 days post 2^{nd} treatment) compared to anti-mCTLA-4 alone. The difference between tumor volume of mice that received the combined treatment and mice treated with an antibody alone was significant. Kaplan-Meier curves revealed a prolonged survival. These results suggest that local short-term treatment with UNO can serve as a treatment option for cancer patients with tumors that are not amenable to checkpoint inhibitor treatment.

### The Effect of Combined Treatment with UNO, a Checkpoint Inhibitor, and an Immune Adjuvant

### Materials and Methods

Preparation of cancer cells for injection—Mouse colon carcinoma CT26.WT cells (ATCC, Cat no. CRL-2638) suspended in Hanks' Balanced Salt Solution (HBSS) (Biological Industries, Israel), at a concentration of 5.0×10⁶ cells/ml, were freshly prepared on the day of cancer model induction. Cells were grown to 70% confluency and were harvested using trypsin (Biological Industries, Israel), and counted using a cell counter device. The cells were then centrifuged at 1,200 rpm (241×g) for 8 minutes, and the pellet was re-suspended in ice cold HBSS at 5.0×10⁶ in 1 ml (5.0×10⁵ cells in 100 μl solution). Cell viability was >90% as determined by trypan blue staining prior to inoculation to mice. At the point of inoculation, cells were at passage 8 at most.

Inoculation of CT26 cancer cells to mice—100 μl/mouse of the_CT26 cancer cells suspension (concentration of 5.0×10⁶ cells/ml) were inoculated subcutaneously (s.c.) to the right flank of male BALB/c mice, which were 10 weeks of age (Envigo, the Netherlands). Five days later, CT26 cells at the same dose were inoculated s.c. to the left flank of each mouse. Ten days post primary tumor inoculation tumor-bearing mice were divided into the following groups (day 0) as depicted in Table 2 below and treated according to the timeline shown in FIG. 20:

TABLE 2

| Treatment | No. of mice |
|---|---|
| Untreated | 10 |
| UNO | 10 |
| UNO + anti-mPD-1 | 10 |
| UNO + anti-mPD-1 + CpG-B | 9 |

Mice were followed up for totally 52 days. Tumor take was monitored by palpation of the flank and tumor growth was monitored by measurement of the tumor dimensions using a digital caliper. Mice were sacrificed and excluded from the study if their total tumor volume was >1500 mm³ or their distress scoring >12 as was accepted according to the IACUC IL-2112-105-5.

UNO treatment—On the day of treatment, mice were anesthetized by an intraperitoneal (i.p.) injection of 100 mg/kg ketamine and 10 mg/kg of xylazine hydrochloride solution. Tumor-bearing mice were treated with 50,000 ppm of UNO delivered into the tumor by a 23 G needle over 5 minutes, at 0.2 LPM. After treatment, all mice were warmed and closely monitored until complete recovery was observed.

Immune checkpoint inhibitor treatment—A murine anti-PD-1 or anti-mPD-1 (BioXcell, Cat No. BP0146) was administered 5 times intraperitoneally (i.p.) at a dose of 10 mg/kg. The injections were administered to approximately the same location each time.

Injection of Class B CpG oligodeoxynucleotide (CpG-B)—CpG-B ODN1826 (IDT, Cat No. 230860406) was administered 3 times s.c. at a dose of 50 μg/0.1 ml in saline. The injections were administered to approximately the same location each time.

### Example 4

### The Effect of Combined Treatment with UNO, Anti-PD-1 and CPG-B on Primary Tumors

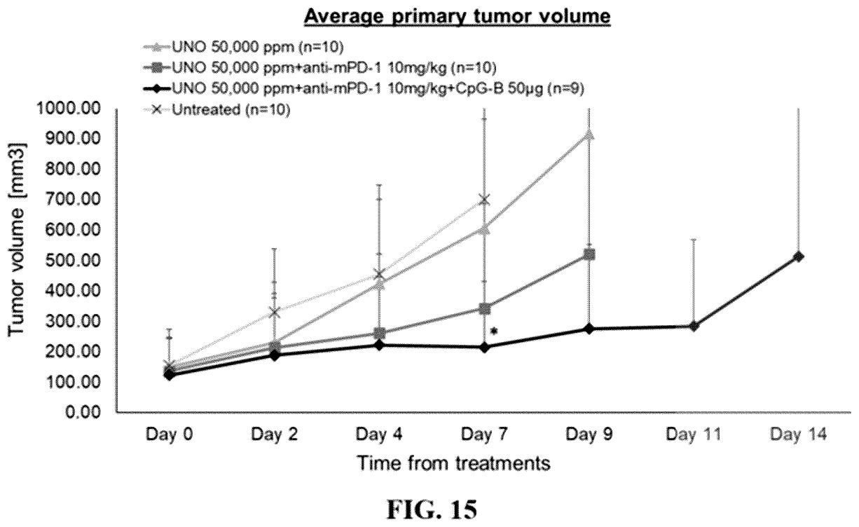

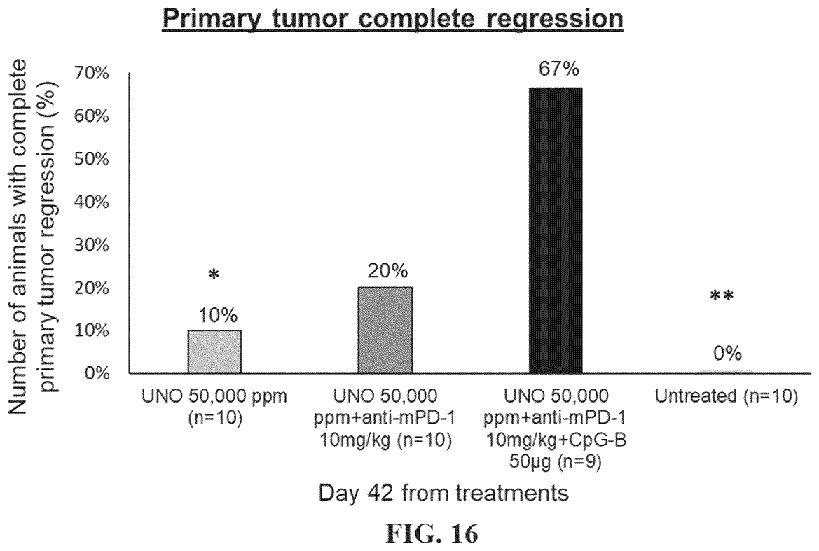

Primary tumors of mice treated with UNO combined with anti-mPD-1 and CpG-B were significantly smaller than tumors of untreated mice or mice treated with UNO alone or UNO+anti-mPD-1 (FIG. 15). Further, the triple combination treatment group showed the highest percentage of primary tumor complete regression (FIG. 16): 6 out of 9 mice (67%), compared to untreated (0%), UNO alone (10%) and UNO+anti-mPD-1 (20%) treated mice.

### Example 5

### The Effect of Combined Treatment with UNO, Anti-PD-1 and CPG-B on Distant Tumor Rejection and Secondary Tumors

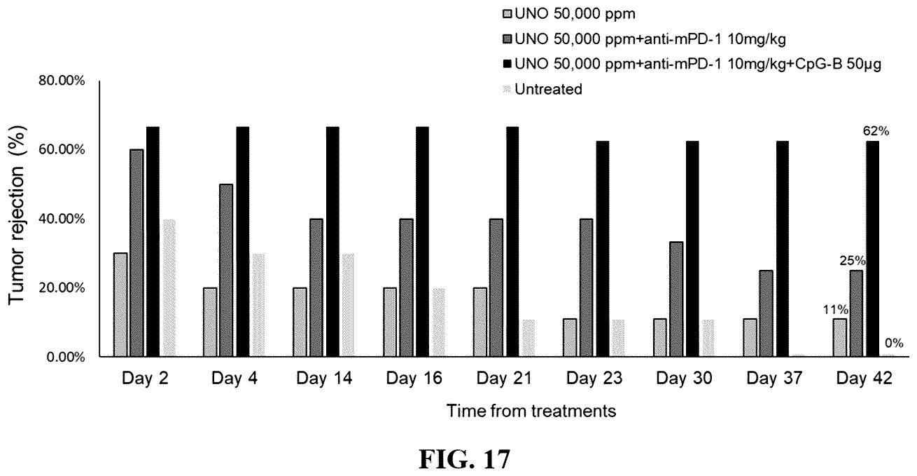

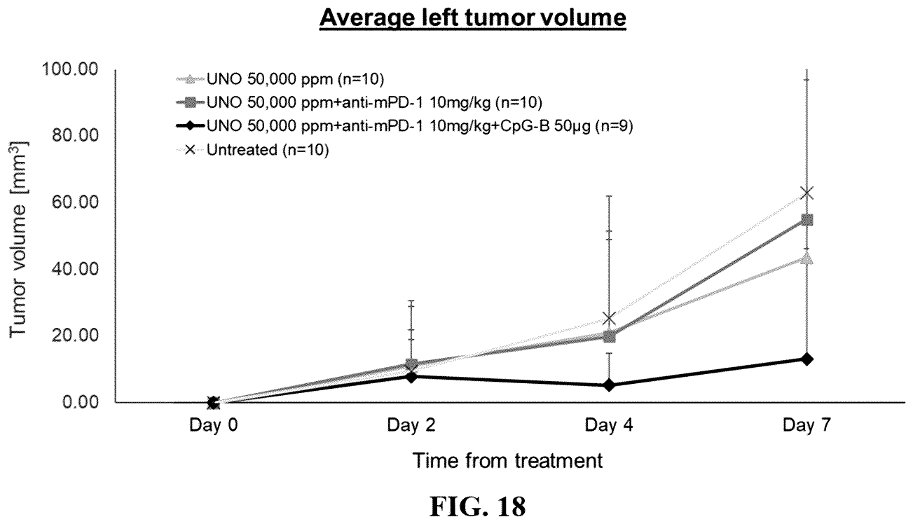

Triple combination treatment with UNO+anti-mPD-1+CpG-B had a significant effect on distant tumor rejection 42 days following treatments (62%) as compared to untreated (0%), UNO alone (11%) and UNO+anti-mPD-1 (25%) treated mice (FIG. 17). Further, the secondary tumors of mice treated with UNO combined with anti-mPD-1 and CpG-B were significantly smaller than the secondary tumors of untreated mice or mice treated with UNO alone or UNO+anti-mPD-1 (FIG. 18).

### Example 6

### The Effect of Combined Treatment with UNO, Anti-PD-1 and CPG-B on Mice Survival

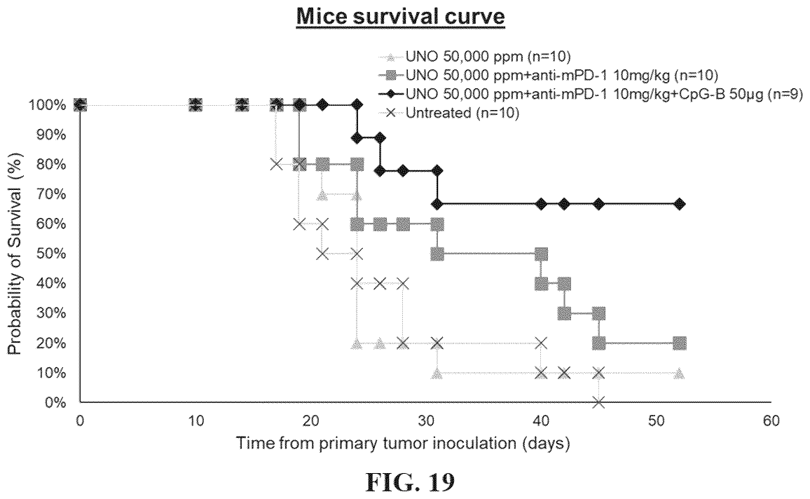

As shown in FIG. 19, the triple combination treatment with UNO+anti-mPD-1+CpG-B had a significant effect on mice survival compared to untreated, UNO alone and UNO+anti-mPD-1 treated mice. Specifically, while the last untreated mouse was sacrificed 45 days post primary tumor inoculation, 10% of the UNO only group, 20% of the UNO+anti-mPD-1 group and 67% of the UNO+anti-mPD-1+CpG-B group survived 52 days following primary tumor inoculation.

### Example 7

### In-Vitro UNO Treatment Increases Presentation of PD-L1 on Cancerous Cells

### Materials and Methods

Preparation of cells—Mouse colon carcinoma CT26.WT cells (ATCC number: CRL-2638) were grown to a 70-80% confluency and harvested using trypsin. Following, the cells were centrifuged at 1,200 revolutions per minute (rpm)—241 G for 8 minutes, then the pellet was re-suspended in 1 ml of cell media (RPMI—1640 supplemented with 10% FBS and 1% Pen-Strep). The cells were counted using an automated cell counter and a total of 2×10⁶ cells were seeded into each 10 mm dish containing 9 ml cell media. The plates were then placed for 24 hours in an incubator at 37° C., 5% CO₂.

UNO treatment—Following 24 hours of incubation, cell media was removed, and the plate was placed in an acrylic box stationed within an operating chemical fume hood. Prior to the initiation of treatment, the box was exposed to UV for 1 hour to reduce the chances of contamination. The box was closed and sealed with parafilm. A PVC delivery line was placed within a designated hole on the top of the box and lowered until approximately 5 cm within the box. The flowmeter was set to 1 LPM to expose the cells for the durations and concentrations described in Table 3 (control cells were untreated).

TABLE 3

| Exposure time | UNO concentrations |
|---|---|
| 10 seconds | 25,000 ppm UNO |
| 30 seconds | 50,000 ppm UNO |
| 1 minute | 100,000 ppm UNO |

Following treatment, 9 ml of cell media were added to each well and the plate was placed for 24 hours in an incubator at 37° C., 5% CO₂.

Flow cytometry—Cell media was removed to a 50 ml centrifuge tube. Each well was washed with 2 ml trypsin, and then the trypsin was collected to the previously described 50 ml centrifuge tube. Following, 3 ml of trypsin was added, and the dish was placed in an incubator at 37° C., 5% CO₂ for 3 minutes. The remaining cells were detached using a cell scraper. 5 ml of cell media was then added to the dish and all of the volume was collected (8 ml) to the centrifuge tube. Subsequently, the cells were centrifuged at 1200 rpm (241 G) for 8 minutes, the supernatant discarded, and the cells were resuspended in 1 ml cell media and stained with Annexin V-FITC kit (Miltenyi Biotec, cat #130-092-052), to detect apoptotic, necrotic, and dead cells or with anti-PD-L1 (Biolegend, Brilliant Violet 421™ anti-mouse CD274 (B7-H1, PD-L1) Antibody, Cat. No. 124315.).
Cell count (MACSQuant VYB): 100 μl from each sample was transferred into a 96 well plate for cell counting purpose. 10⁶ cells were distributed to a designated Eppendorf tube for labeling purposes.
Cell Staining with anti-mouse CD274 (B7-H1, PD-L1) BV421 antibody (BioLegend, Cat. No. 124315) and Rat IgG2b, Isotype control antibody (BioLegend, Cat. No. 400639): each sample was stained with 5 μl of anti-mouse CD274 antibody in 95 μl of FACS Buffer (100 μl total labeling volume). Each isotype control sample was stained with 5 μl of Rat IgG2b, Isotype control antibody in 95 μl of FACS Buffer. Unstained samples were resuspended in 100 μl of FACS Buffer. The cells were incubated for 20 minutes at 4° C., covered in aluminum foil. Cells were washed by adding 500 μl of FACS Buffer and centrifuged for 7 minutes at 300×g.
Cell Staining with Annexin V FITC Kit from Miltenyi (Cat. No. 130-092-052): The supernatant was discarded, and the cells were washed with 1.5 ml of 1×Binding Buffer by centrifugation at 300×g for 7 minutes. Annexin V Staining Solution was prepared by diluting the Annexin V FITC Reagent 1:11 with the 1×Binding Buffer. The cells were resuspended in 110 μl of Annexin V FITC Solution (except PI single stain and unstained samples which were resuspended in 110 μl of 1×Binding Buffer). The samples were incubated for 15 minutes at room temperature protected from light. The cells were washed by adding 1 ml of 1×Binding Buffer and centrifuged at 300×g for 7 minutes at room temperature. The samples were resuspended in 500 μl of 1×Binding Buffer.
Acquisition on MACSQuant VYB flow cytometer: 5 μl of PI was added immediately prior to analysis on MACSQuant VYB instrument.

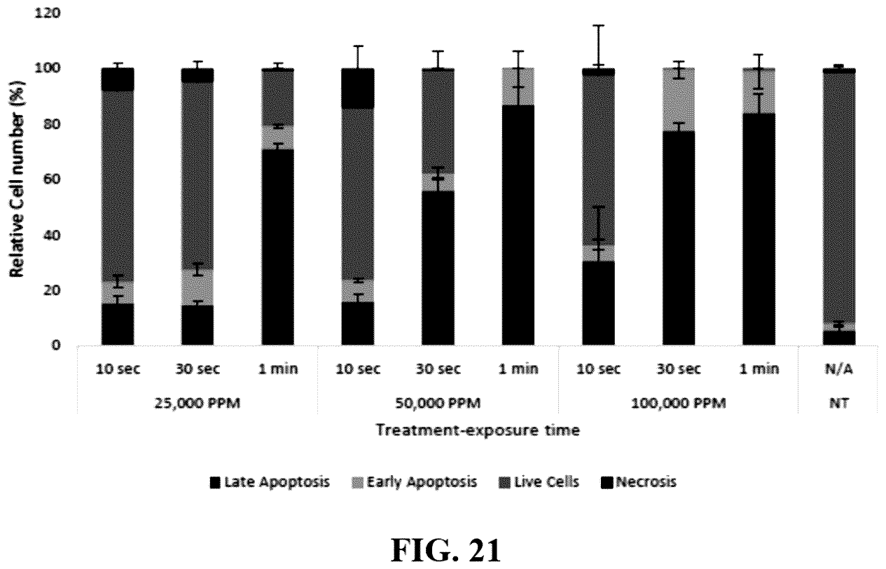

The effect of exposure to UNO on viability of CT26 cancer cells was assessed 24 hours following exposure. As shown in FIG. 21, exposure to UNO reduces viability and induces apoptosis and necrosis of the cancer cells in a dosage and time dependent manner.

Interestingly, following exposure to UNO about 70-90% of the PI negative cells expressed PD-L1, depending on the dosage and time of exposure (FIG. 22). Furthermore, the mean fluorescent intensity (MFI) of PD-L1 expression increased in a UNO dosage and time dependent manner. As such, by upregulating the PD-L1 expression on the tumor, the tumor can become more responsive to anti-PD-1 and/or anti-PD-L1 treatment. Accordingly, the UNO can be used to sensitize the tumor before the anti-PD-1 or anti-PD-L1 treatment.

### Example 8

### The Effects of UNO and Anti-CTLA-4 on Systemic Levels of Tumor Antigen-Specific CD8+ T-Cells 7 Days after Treatment

### Materials and Methods

Preparation of cancer cells for injection—Mouse colon carcinoma CT26.WT cells (ATCC, Cat no. CRL-2638) suspended in Hanks' Balanced Salt Solution (HBSS) (Biological Industries, Israel), at a concentration of 5.0×10⁶ cells/ml, were freshly prepared on the day of cancer model induction. Cells were grown to 70% confluency and were harvested using trypsin (Biological Industries, Israel), and counted using a cell counter device. The cells were then centrifuged at 1,200 rpm (241×g) for 8 minutes, and the pellet was re-suspended in ice cold HBSS at 5.0×10⁶ in 1 ml (5.0×10⁵ cells in 100 μl solution). Cell viability was >90% as determined by trypan blue staining prior to inoculation to mice. At the point of inoculation, cells were at passage 8 at most.

Inoculation of CT26 cancer cells to mice—100 μl/mouse of the CT26 cancer cells suspension (concentration of 5.0×10⁶ cells/ml) were inoculated subcutaneously (s.c.) to the right flank of male BALB/c mice, which were 10 weeks of age (Envigo, the Netherlands). Three days later, CT26 cells at the same dose were inoculated s.c. to the left flank of each mouse. Ten days after the primary tumor inoculation, the tumor-bearing mice were divided into the following groups (day 0) as depicted in Table 4 below:

TABLE 4

| | No. of |
|---|---|
| Treatment | mice |
| Untreated | 8 |
| UNO | 8 |
| anti-mCTLA-4 | 8 |
| UNO + anti-mCTLA-4 | 8 |

UNO treatment—On the day of treatment, mice were anesthetized by an intra-peritoneal (i.p.) injection of 100 mg/kg ketamine and 10 mg/kg of xylazine hydrochloride solution. Tumor-bearing mice were treated with 100,000 ppm of UNO delivered into the tumor by a 23 G needle over 5 minutes, at 0.2 LPM. After treatment, all mice were warmed and closely monitored until complete recovery was observed.

Immune checkpoint inhibitor treatment—A murine anti-CTLA-4 or anti-mCTLA-4 (BioXcell, Cat No. BE-0131) was administered 2 times intraperitoneally (i.p.) at a dose of 5 mg/kg. The injections were administered to approximately the same location each time.

Flow cytometry for tumor antigen-specific CD8+ T-cells—On day 7 after the UNO treatment, 100 μl of peripheral blood was collected by submandibular bleeding from each mouse into tubes containing 0.5M EDTA PH 8.0. Red blood cells were removed by incubation in ACK lysis solution for 5 minutes, and samples were centrifuged for 5 minutes at 1500 RPM (300 g). Samples were subsequentially stained with 1 μl/ml ghost dye Red 710 (TONBO biosciences, Cat No. 13-0871) for 15 minutes at 4° C. and washed two times with FACS buffer containing 2% Fetal bovine serum in PBS. Samples were then stained for 30 minutes at room temperature with a mix of: 0.5 μg/ml CD8-FITC Antibody (Invitrogen, Clone KT15, Cat No. MA516759), 0.66 μg/ml CD4-APC/Fire810 Antibody (Biolegend, Clone GK1.5, Cat No. 100480), 3 μg/ml CD3-PerCP-Vio700 Antibody (Miltenyi Biotec, Clone REA641, Cat No. 130-120-826), 0.75 μg/ml CD44-PE-vio770 Antibody (Miltenyi Biotec, Clone REA664, Cat No. 130-119-127), and 0.75 μg/ml CD62L-APC Antibody (Miltenyi Biotec, Clone REA828, Cat No 130-112-837) and 4 μl/sample of H-2LdMuLVgp70 Tetramer (MBL Life science, Cat No. TB-M521-1). Samples were then washed two times with FACS buffer and acquired on an Attune NxT flow cytometer. Data was analyzed using the FlowJo software.

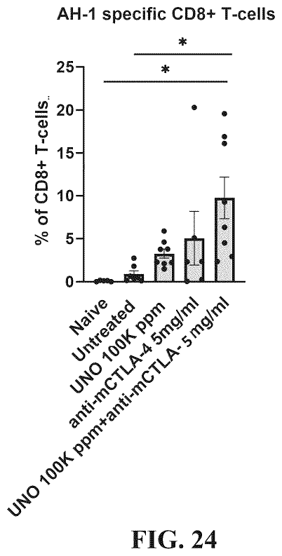

Results: AH-1+CD8+ T-cells (tumor antigen-specific CD8+ T-cells) were detected in the blood of all mice treated with UNO but not in mice treated with anti-mCTLA-4, untreated or naïve mice (FIG. 24). Furthermore, systemic levels of AH-1+CD8+ T-cells levels were higher in mice treated with UNO compared to untreated or naïve mice that did exhibit AH-1+CD8+ T-cells. In addition, AH-1+CD8+ T-cells were found in the blood of all mice treated with the combination of UNO and anti-mCTLA-4 and to a higher level compared to mice treated with anti-mCTLA-4 or UNO alone.

### Example 9

The treatment of cutaneous or subcutaneous primary or metastatic tumors using UNO at a dose of 50,000 ppm administered intratumorally will be studied. The UNO will be administered for a duration of 5 minutes at a flow rate of 0.2 L/min. The UNO will be administered for multiple 21-day cycles, with the UNO being administered on day 1 of each cycle, e.g., every 21 days.

### Example 10

The treatment of triple negative breast cancer (TNBC) using a combination of (i) UNO at a dose of 50,000 ppm administered intratumorally for a duration of 5 minutes at a flow rate of 0.2 L/min and (ii) a PD-1 inhibitor, pembrolizumab, at a dose of 200 mg or 400 mg administered intravenously for a duration of 30 minutes, with the UNO being administered immediately prior to the PD-1 inhibitor will be studied. The UNO will be administered for multiple 21-day cycles, with the UNO being administered on day 1 of each cycle, e.g., every 21 days.

### Example 11

The treatment of advanced cutaneous malignant melanoma using a combination of (i) UNO at a dose of 25,000 ppm or 50,000 ppm administered intratumorally for a duration of 5 minutes and a flow rate of 0.2 L/min and (ii) a CTLA-4 inhibitor, ipilimumab, at a dose of 3 mg/kg administered intravenously for a duration of 30 minutes, with the UNO being administered immediately prior to the CTLA-4 inhibitor will be studied. The combination will be administered for multiple 21-day cycles, with the combination being administered on day 1 of each cycle, e.g., every 21 days.

While this invention has been particularly shown and described with references to preferred embodiments thereof, it will be understood by those skilled in the art that various changes in form and details may be made therein without departing from the scope of the invention encompassed by the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of treating cancer in a subject in need thereof, the method comprising administering to the subject a therapeutically effective amount of ultra-high concentration gaseous nitric oxide (UNO) and a checkpoint inhibitor, wherein the UNO is administered intratumorally.

2. (canceled)

3. The method of claim 1, wherein said UNO is administered at a dose of about 10,000 ppm to about 1,000,000 ppm for a time period of from about 1 second to about 60 minutes at a volumetric flow of from about 0.00001 LPM to about 1 LPM.

4. The method of claim 1, wherein said UNO is administered at a dose of about 20,000 ppm to about 200,000 ppm or about 20,000 ppm to about 100,000 ppm.

5. The method of claim 1, wherein said UNO is administered for a time period that ranges from about 30 seconds to about 10 minutes.

6. The method of claim 1, wherein said UNO is administered at a volumetric flow of about 0.001 LPM to about 0.5 LPM.

7. The method of claim 1, wherein said checkpoint inhibitor is administered prior to said UNO.

8. The method of claim 1, wherein said checkpoint inhibitor is administered every 2-7 days.

9. The method of claim 1, wherein said checkpoint inhibitor is administered at least twice.

10. The method e of claim 1, wherein said UNO is administered prior to said checkpoint inhibitor.

11. The method of claim 1, wherein the administration of UNO sensitizes the cancer to treatment by the checkpoint inhibitor by upregulating the expression of a target immune checkpoint protein before the administration of the checkpoint inhibitor, wherein the target immune checkpoint protein is one of PD-1, PD-L1, and CTLA-4.

12. The method of claim 1, wherein the combination of UNO and the checkpoint inhibitor results in an increase of tumor-specific immune cells.

13. The method e of claim 1, wherein the checkpoint inhibitor is one a PD-1 inhibitor, a PD-L1 inhibitor, a CTLA-4 inhibitor.

14. The method of claim 1, wherein said cancer is refractory to treatment with the checkpoint inhibitor.

15. The method of claim 1, further comprising administering an immune adjuvant.

16. The method of claim 15, wherein said immune adjuvant is selected from the group consisting of mineral salt, aluminum salt, an organic adjuvant, emulsion, microparticle, liposome, saponin, cytokine, microbial component and a nucleic acid adjuvant.

17. The method of claim 16, wherein said immune adjuvant is a nucleic acid adjuvant.

18. The method of claim 17, wherein said nucleic acid adjuvant comprises a cytosine-phosphorothioate-guanine oligodeoxynucleotide (CpG ODN).

19. The method of claim 17, wherein said checkpoint inhibitor is administered prior to said immune adjuvant.

20. The method of claim 17, wherein said immune adjuvant is administered subsequent to said UNO.

21. The method of claim 1, wherein said cancer is positive for a microsatellite instability (MSI) and/or a mismatch repair deficient (dMMR) marker.

22. The method of claim 1, wherein said cancer is negative for a microsatellite instability (MSI) and/or a mismatch repair deficient (dMMR) marker.

23. The method of claim 1, wherein said cancer is selected from the group consisting of colon, breast, melanoma, lung, Head and Neck Squamous Cell Cancer (HNSCC), Classical Hodgkin Lymphoma (cHL), Primary Mediastinal Large B-Cell Lymphoma (PMBCL), Urothelial Carcinoma, Gastric Cancer, Esophageal Cancer, Cervical Cancer, Hepatocellular Carcinoma (HCC), Merkel Cell Carcinoma (MCC), Renal Cell Carcinoma (RCC), Endometrial Carcinoma, Tumor Mutational Burden-High (TMB-H) Cancer, Cutaneous Squamous Cell Carcinoma (cSCC), Triple-Negative Breast Cancer (TNBC), Microsatellite Instability-High or Mismatch Repair Deficient Cancer and Microsatellite Instability-High or Mismatch Repair Deficient Colorectal Cancer (CRC).
